(19) United States
(12) Patent Application Publication
(10) Pub. No.: US 2019/0219580 A1
(43) Pub. Date: Jul. 18, 2019

(54) METHODS OF DRUG THERAPY SELECTION FOR BREAST CANCER PATIENTS BASED ON HER2 AND HER3 PATHWAY SUBTYPING

(71) Applicant: Nestec S.A., Vevey (CH)

(72) Inventors: Joseph P. Michel, San Diego, CA (US); Emma Langley, San Diego, CA (US); Phillip Kim, San Diego, CA (US)

(73) Assignee: Nestec S.A., Vevey (CH)

(21) Appl. No.: 16/202,799

(22) Filed: Nov. 28, 2018

Related U.S. Application Data
(63) Continuation of application No. PCT/US2017/035045, filed on May 30, 2017.
(60) Provisional application No. 62/343,555, filed on May 31, 2016.

(51) Int. Cl. G01N 33/574; C07K 16/32; A61P 35/00

(52) U.S. Cl. CPC G01N 33/57415; C07K 16/32; A61K 2039/505; C07K 2317/24; A61P 35/00

(57) ABSTRACT

Provided herein is a method for determining whether a human subject with breast cancer will respond to a therapy comprising a tyrosine kinase inhibitor or a biologic. The method includes determining the expression level and/or activation level of various signal transduction molecules such as truncated HER2 protein, full-length HER2 protein, HER3 protein, PI3K protein, and others. The determination of likely response to a tyrosine kinase inhibitor therapy or a biologic therapy involves comparing the expression level and/or activation level of the signal transduction molecule(s) to a reference expression/activation level for the specific signal transduction molecule(s).

### CROSS-REFERENCE TO RELATED APPLICATION

The present application is a continuation of PCT Application No. PCT/US2017/035045, filed on May 30, 2017, which claims priority to U.S. Provisional Application No. 62/343,555, filed May 31, 2016, the disclosures of which are hereby incorporated by reference in their entirety for all purposes.

### BACKGROUND OF THE INVENTION

HER2 overexpression, which occurs in approximately 25% of breast cancers, results in increased cell proliferation and is associated with poor clinical outcome. Trastuzumab, a recombinant humanized monoclonal antibody against the extracellular domain of the HER2 protein, was developed to block HER2 signaling pathways and has been shown to substantially improve the efficacy of chemotherapy in women with metastatic and early-stage HER2-positive breast cancers. A joint analysis of the NSABP B-31 and NCCTG N9831 studies showed a 52% improvement in disease-free survival (DFS) with incorporation of trastuzumab into an adjuvant regimen of doxorubicin and cyclophosphamide (AC)→paclitaxel in women with HER2-positive, operable breast cancer. The BCIRG 006 study showed similar reduction in risk for DFS events by adding trastuzumab to a sequential regimen of AC→docetaxel. Trastuzumab given after completion of adjuvant or neoadjuvant chemotherapy (HERA Trial) also provided improvement in outcome. On the basis of these results, sequential AC followed by a taxane initiated concurrently with trastuzumab has become a standard of care in the United States for operable HER2-positive breast cancer following initial surgery.

The human epidermal growth factor receptors are members of the HER receptor kinase family which includes four receptors: EGFR/HER1/ErbB1, HER2/ErbB2, HER3/ErbB3, and HER4/ErbB4. All four receptors consist of an extracellular binding domain, a single membrane spanning region, and regulatory domains. HER1, HER2 and HER4 have an intracellular tyrosine-kinase domain, HER3 does not. HER2 is ligandless but functions as a coreceptor and is actually the preferred partner for the other ErbB family members. The heterodimer HER2:HER3 has the most potent downstream signaling. Ligand binding induces the formation of multiple combinations of ErbB receptor homo- and heterodimers, resulting in activation of the cytoplasmic kinase domain. This is turn promotes the phosphorylation of specific tyrosine residues, leading to the stimulation of multiple signal transduction pathways.

Hyperactivation of ErbB receptors may occur because of overexpression or by ligand-mediated stimulation. Trastuzumab which binds at domain IV of HER2 is thought to have multiple possible mechanisms by which it may exert its antiproliferative and therapeutic function including inhibition of the signaling function of ligand-independent HER2 homodimerization by blocking dimerization-dependent activation of HER2 with HER1 or HER3 and by blocking HER2-Src interaction. As trastuzumab is an intact monoclonal antibody, the Fcγ portion of the molecule may play a significant role in the in vivo activity by its ability to engage Fcγ receptors on immune effector cells such as macrophages, NK cells or cytotoxic T cells in order to elicit antibody-dependent cellular cytotoxicity (ADCC). A recent clinical finding is that Fcγ receptor polymorphisms may be determinants of trastuzumab response in breast cancer patients and supports the potential role of ADCC in trastuzumab-based therapies

Patients with metastatic HER2-positive disease will eventually become resistant to trastuzumab-based therapies. There are multiple proposed mechanisms of resistance to trastuzumab including PI3K hyperactivation from upstream signaling through heregulin-induced ligand stimulation, alternate growth factor receptor heterodimerization such as HER2:HER3, and polymorphisms of the Fcγ receptors that reduce the binding to immune effector cells. Pertuzumab, another antibody to HER2, binds to domain II and prevents the heterodimerization of HER2:HER3. A Phase II trial of pertuzumab and trastuzumab (n=66) in patients with HER2-positive metastatic breast cancer that progressed during prior trastuzumab had an overall response rate of 24% and complete response rate of 7.6%. The median progression-free survival was 5.5 months. In a Phase III study (n=808) in metastatic disease, dual inhibition with trastuzumab and pertuzumab plus docetaxel resulted in improved progression-free survival compared to trastuzumab and docetaxel alone (12.4 vs. 18.5 months, HR 0.62, p<0.001).

In the NeoSphere study (n=417), trastuzumab and pertuzumab, either alone or combined or added to docetaxel, were evaluated preoperatively in women with locally advanced and inflammatory breast cancer. The pathologic complete responses (pCR) were as follows: trastuzumab plus docetaxel (TH), 29%; trastuzumab, pertuzumab plus docetaxel (THP), 46%; trastuzumab and pertuzumab (HP), 17%; and pertuzumab plus docetaxel (TP), 24%. Pathologic CR with THP was significantly higher (p=0.014) than with TH which was significantly higher than with HP (p=0.020). Taken together, the interpretation of these studies suggests that a more complete blockage of the HER pathway resulted in a higher pCR.

Another potential way to block downstream signaling is to inhibit tyrosine kinase activity of dimers such as HER1:1, HER1:2, HER1:3 and HER2:3 with a small molecule inhibitor such as lapatinib or neratinib. Lapatinib has demonstrated activity as first-line therapy in patients with HER2-positive metastatic and locally advanced breast cancer with an overall response rate of 24%. Lapatinib's approval was based on its activity combined with capecitabine in women with progressive, locally advanced, or metastatic HER2-positive breast cancer previously treated with an anthracycline, a taxane, and trastuzumab. The treatment regimen of an anthracycline, a taxane, and trastuzumab demonstrated an overall response rate of 22% with an improvement in median TTP from 4.4 months with capecitabine alone to 8.4 months with capecitabine plus lapatinib (HR, 0.49; 95% CI, 0.34 to 0.71; p=0.00004, log-rank, 1-sided).

The NeoALLTO trial randomized patients (n=450) to lapatinib plus paclitaxel, versus trastuzumab plus paclitaxel versus concomitant lapatinib and trastuzumab plus paclitaxel. In the concomitant arm of lapatinib and trastuzumab plus paclitaxel, the dose of lapatinib was reduced from 1000 mg/day to 750 mg/day because of GI toxicity. In the combination arm, the study met its primary endpoint with a pCR(breast) of 51.3% compared to 29.5% and 24.7% respectively in the trastuzumab and lapatinib arms alone. The Geparquinto study (n=620) in neoadjuvant, HER2-positive patients compared epirubicin, cyclophosphamide followed by docetaxel (EC-DOC) with either trastuzumab or lapatinib. The pCR rate (no invasive disease in breast) favored the trastuzumab arm, 50% vs. 35% (p<0.05). Of note, the lapatinib dose was lowered due to toxicity in the run-in phase and more patients discontinued targeted therapy on lapatinib.

In another study presented at the San Antonio Breast Cancer Symposium in 2008, results from two cohorts of patients with HER2-positive, locally advanced disease without previous treatment were reported. The first study of 40 patients employed trastuzumab weekly×3 cycles followed by the combination of trastuzumab and docetaxel every 3 weeks×4 cycles. The pCR rate was 34%. In the second cohort of 49 patients, lapatinib was given as a single agent at a full dose of 1500 mg daily for 6 weeks followed by trastuzumab/docetaxel×4 cycles. The pCR rate in this cohort was 68%. This study included a provocative analysis of biomarkers that showed in patients with low PTEN expression and PI3 kinase mutation. The pCR rate in the trastuzumab cohort was 18%, while the pCR rate was 87% with lapatinib, suggesting differing mechanisms of action. Potential mechanisms of resistance to trastuzumab include: cleavage of the extracellular domain of HER2, which results in a potent oncogenic receptor (p95HER2) that is less responsive to trastuzumab; abnormal PTEN function; and heterodimerization with EGFR with continued activation through the heterodimer by EGFR activation in spite of interruption of HER2 signaling.

Lapatinib and neratinib both bind to the ATP sites of pan ErbB receptor tyrosine kinases and prevent phosphorylation and activation of downstream signaling pathways. In BT474 cell lines, neratinib (HKI-272) effectively repressed phosphorylation of MAPK and AKT signal transduction pathways, whereas trastuzumab failed to completely inhibit HER2 receptor phosphorylation or downstream signaling events. In tumor xenografts which overexpress HER2, neratinib has been observed to repress tumor growth in a dose-dependent manner

Overall response rates with lapatinib and neratinib in comparable patients, albeit in separate Phase II studies, suggest favorable efficacy of neratinib as monotherapy in trastuzumab-refractory patients (response rate of 5.1% vs. 26%) and in trastuzumab-naive patients (response rate of 24% vs. 56%). Taken together, the data show that therapy selection to treat patients with breast cancer is challenging. As such, there is a need in the art for improved methods of drug therapy selection for breast cancer patients.

### BRIEF SUMMARY OF THE INVENTION

In one aspect, the present invention provides a method for determining whether a human subject with breast cancer will respond to therapy with a tyrosine kinase inhibitor or a biologic. The method includes: (a) lysing a breast cancer cell obtained from a sample from the human subject to produce a cellular extract; (b) determining an expression level of truncated HER2 protein, an expression level of full-length HER2 protein, an activation level of full-length HER2 protein, an activation level of HER3 protein, and/or an activation level of PI3K protein in the cellular extract; (c) comparing the expression level of truncated HER2 protein, the expression level of full-length HER2 protein, the activation level of full-length HER2 protein, the activation level of HER3 protein, and/or the activation level of PI3K protein in the cellular extract to a reference expression level of truncated HER2 protein, a reference expression level of full-length HER2 protein, a reference activation level of full-length HER2 protein, a reference activation level of HER3 protein, and/or a reference activation level of PI3K protein, respectively; and (d) determining whether a human subject with breast cancer will respond to therapy with a tyrosine kinase inhibitor or a biologic based upon a difference between the expression level of truncated HER2 protein, the expression level of full-length HER2 protein, the activation level of full-length HER2 protein, the activation level of HER3 protein, and/or the activation level of PI3K protein in the cellular extract compared to the reference expression level of truncated HER2 protein, the reference expression level of full-length HER2 protein, the reference activation level of full-length HER2 protein, the reference activation level of HER3 protein, and/or the reference activation level of PI3K protein, respectively.

Other objects, features, and advantages of the present invention will be apparent to one of skill in the art from the following detailed description and figures.

### BRIEF DESCRIPTION OF THE DRAWINGS

FIG. 1 provides the design of the clinical study described in Example 1.

FIGS. 2A and 2B provides information about the tumor samples (FIG. 2A) and signal transduction molecules (FIG. 2B) analyzed in the CEER study.

FIGS. 3A-3C show the distribution of HER2 expression in baseline tumor samples from patients with stage IIB to III breast cancer. These patients were also HER positive. FIG. 3A represents the data on a linear scale. FIG. 3B represents the data on a natural log scale. FIG. 3C provides the distribution in terms of quantiles and quartiles.

FIGS. 4A and 4B show the correlation between the presence of pathological complete response and the distribution of HER2 expression in the baseline tumor samples.

FIGS. 5A-5C show the levels of p95HER2 expression in baseline tumor samples in non-responders to neratinib, responders to neratinib, non-responders to trastuzumab, and responders to trastuzumab. FIG. 5A represents the level of p95HER2 expression as a ratio of the expression level of p95HER2 protein to the expression level of cytokeratin (p-value=0.006 for neratinib non-responder vs. neratinib responder; p-value=0.027 for neratinib responder vs. trastuzumab non-responder). FIG. 5B provides the ratios of the 75% quartile, median, and 25% quartile in each of the groups analyzed. FIG. 5C shows the percent fold change of the median levels of total p95HER2 protein in the groups analyzed.

FIGS. 6A-6C show the levels of full-length HER2 protein in baseline tumor samples in non-responders to neratinib, responders to neratinib, non-responders to trastuzumab, and responders to trastuzumab. FIG. 6A represents the level of total HER2 protein as a ratio of the expression level of HER2 protein to the expression level of cytokeratin (p-value=0.027 for neratinib non-responder vs. neratinib responder; p-value=0.032 for neratinib responder vs. trastuzumab non-responder). FIG. 6B provides the ratios of the 75% quartile, median, and 25% quartile in each of the groups analyzed. FIG. 6C shows the percent fold change of the median levels of total HER2 protein in the groups analyzed.

FIGS. 7A-7C show the levels of activated HER2 protein in baseline tumor samples in non-responders to neratinib, responders to neratinib, non-responders to trastuzumab, and responders to trastuzumab. FIG. 7A represents the level of activated HER2 protein as a ratio of the activation level of HER2 protein to the expression level of cytokeratin (p-value=0.07 for neratinib responder vs. trastuzumab non-responder). FIG. 7B provides the ratios of the 75% quartile, median, and 25% quartile in each of the groups analyzed. FIG. 7C shows the percent fold change of the median levels of activated HER2 protein in the groups analyzed.

FIGS. 8A-8C show the levels of activated HER3 protein in baseline tumor samples in non-responders to neratinib, responders to neratinib, non-responders to trastuzumab, and responders to trastuzumab. FIG. 8A represents the level of activated HER3 protein as a ratio of the activation level of HER3 protein to the expression level of cytokeratin (p-value=0.025 for neratinib non-responder vs. trastuzumab responder; p-value=0.07 for neratinib responder vs. trastuzumab responder; p-value=0.11 for trastuzumab non-responder vs. trastuzumab responder).

FIG. 8B provides the ratios of the 75% quartile, median, and 25% quartile in each of the groups analyzed. FIG. 8C shows the percent fold change of the median levels of activated HER3 protein in the groups analyzed.

FIGS. 9A-9C show the levels of activated PI3K protein in baseline tumor samples in non-responders to neratinib, responders to neratinib, non-responders to trastuzumab, and responders to trastuzumab. FIG. 9A represents the level of activated PI3K protein as a ratio of the activation level of PI3K protein to the expression level of cytokeratin (p-value=0.07 for neratinib responder vs. trastuzumab responder). FIG. 9B provides the ratios of the 75% quartile, median, and 25% quartile in each of the groups analyzed. FIG. 9C shows the percent fold change of the median levels of activated PI3K protein in the groups analyzed.

FIG. 10 shows the levels of activated HER2 protein, activated ERK protein, activated RSK protein, activated HER3 protein, activated AKT protein, activated PRAS40 protein, and activated RPS6 protein in baseline tumor samples in non-responders to neratinib, responders to neratinib, non-responders to trastuzumab, and responders to trastuzumab.

FIGS. 11A-11C show the expression levels of truncated HER2 protein and the activation level of HER3 protein in baseline tumor samples from non-responders and responders can be combined into a predictive model that can be used to treatment selection. FIG. 11A shows the responders and non-responders can be classified into two separate and distinct groups based on an algorithm containing expression level and activation level data. FIG. 11B shows that the responders and non-responders included only HER2 positive breast cancer patients. FIG. 11C shows sensitivity and specificity of the predictive model.

### DETAILED DESCRIPTION OF THE INVENTION

### I. INTRODUCTION

Provided herein is a method that can aid in treatment selection for patients with breast cancer. Also provided is a method for treating breast cancer by administering a tyrosine kinase inhibitor (TKI) to a patient who is likely to respond to the TKI. Similarly, a method for treating breast cancer by administering a biologic to a patient who is likely to respond to the biologic is provided.

### II. DEFINITIONS

As used herein, the following terms have the meanings ascribed to them unless specified otherwise.

The term “tyrosine kinase inhibitor” or “TKI” refers to any small molecule compound or biologic drug that inhibits (e.g., blocks, disrupts, or inactivates) the activity of a tyrosine kinase (e.g., a receptor tyrosine kinase or a non-receptor tyrosine kinase). For instance, the inhibitor can directly bind (e.g., complex) to a tyrosine kinase, and interrupt (e.g., block) the signal transduction pathway lying downstream of a tyrosine kinase.

The term “biologic,” “biologic drug,” or “biological drugs” refers to a therapeutic agent or drug that is manufactured in, extracted from, derived form, or synthesized in part from a biological source, e.g., human, animal, micro-organism and the like. Non-limiting examples of biologics include blood and components thereof, allergenic extracts, human cells and tissues, vaccines, antibodies, recombinant polypeptides, recombinant polynucleotides, gene therapies, cell therapies, etc. Biologics that can be used to treat cancer include targeted cancer therapies such as humanized antibodies, monoclonal antibodies, immunotherapies, and the like.

The term “neoadjuvant therapy” refers to a preoperative or primary therapy that is administered before surgery.

The term “analyte” refers to any molecule of interest, typically a macromolecule such as a polypeptide, whose presence, amount (expression level), activation state or level, and/or identity is determined. A non-limiting analyte includes a signal transduction molecule including proteins and other molecules that carry out a process by which a cell converts an extracellular signal or stimulus into a response, typically involving ordered sequences of biochemical reactions inside the cell. In certain instances, the analyte is a cellular component of a tumor cell, preferably a molecule of the HER1, HER2 and/or HER3 signaling pathways and other signaling pathways associated with cancer.

The term “signal transduction molecule” or “signal transducer” includes proteins and other molecules that carry out the process by which a cell converts an extracellular signal or stimulus into a response, typically involving ordered sequences of biochemical reactions inside the cell. Examples of signal transduction molecules include, but are not limited to, receptor tyrosine kinases such as EGFR (e.g., EGFR/HER-1/ErbB1, HER-2/Neu/ErbB2, HER-3/ErbB3, HER-4/ErbB4), VEGFR-1/FLT-1, VEGFR-2/FLK-1/KDR, VEGFR-3/FLT-4, FLT-3/FLK-2, PDGFR (e.g., PDGFRA, PDGFRB), c-KIT/SCFR, INSR (insulin receptor), IGF-IR, IGF-IIR, IRR (insulin receptor-related receptor), CSF-1R, FGFR 1-4, HGFR 1-2, CCK4, TRK A-C, MET, RON, EPHA 1-8, EPHB 1-6, AXL, MER, TYRO3, TIE 1-2, TEK, RYK, DDR 1-2, RET, c-ROS, V-cadherin, LTK (leukocyte tyrosine kinase), ALK (anaplastic lymphoma kinase), ROR 1-2, MUSK, AATYK 1-3, RTK 106, and truncated forms of the receptor tyrosine kinases such as p95ErbB2; non-receptor tyrosine kinases such as BCR-ABL, Src, Frk, Btk, Csk, Abl, Zap70, Fes/Fps, Fak, Jak, Ack, and LIMK; tyrosine kinase signaling cascade components such as Akt, MAPK/ERK, MEK, RAF, PLA2, MEKK, JNKK, JNK, p38, Shc (p66), PI3K, Ras (e.g., K-Ras, N-Ras, H-Ras), Rho, Rac1, Cdc42, PLC, PKC, p70 S6 kinase, p53, cyclin D1, STAT1, STAT3, PIP2, PIP3, PDK, mTOR, BAD, p21, p27, ROCK, IP3, TSP-1, NOS, PTEN, RSK 1-3, JNK, c-Jun, Rb, CREB, Ki67, and paxillin; nuclear hormone receptors such as estrogen receptor (ER), progesterone receptor (PR), androgen receptor, glucocorticoid receptor, mineralocorticoid receptor, vitamin A receptor, vitamin D receptor, retinoid receptor, thyroid hormone receptor, and orphan receptors; nuclear receptor coactivators and repressors such as amplified in breast cancer-1 (AIB1) and nuclear receptor corepressor 1 (NCOR), respectively; and combinations thereof.

The term “activation level,” in the context of an analyte or biomarker, refers to the extent of level a particular signal transduction molecule or analyte is activated (e.g., phosphorylated, ubiquitinated, and/or complexed).

The term “expression level,” in the context of an analyte or biomarker, refers to the amount or level of a particular signal transduction protein.

The term “sample” includes any biological specimen obtained from a patient. Samples include, without limitation, whole blood, plasma, serum, red blood cells, white blood cells (e.g., peripheral blood mononuclear cells), saliva, urine, stool (i.e., feces), sputum, bronchial lavage fluid, tears, nipple aspirate, lymph (e.g., disseminated tumor cells of the lymph node), fine needle aspirate, any other bodily fluid, a tissue sample (e.g., tumor tissue) such as a biopsy of a tumor (e.g., needle biopsy), and cellular extracts thereof. In some embodiments, the sample is whole blood or a fractional component thereof such as plasma, serum, or a cell pellet. In certain instances, the sample is obtained by isolating circulating cells of a solid tumor from whole blood or a cellular fraction thereof using any technique known in the art and preparing a cellular extract of the circulating cells. In other embodiments, the sample is a formalin fixed paraffin embedded (FFPE) tumor tissue sample, e.g., from a solid tumor of the breast.

The term “subject” or “patient” typically includes humans, but can also include other animals such as, e.g., other primates, rodents, canines, felines, equines, ovines, porcines, and the like.

The phrase “1-fold higher” or “100% fold change higher,” in the context of an analyte, refers to a level (e.g., expression level or activation level) that is double the reference level of the analyte.

### III. DETAILED DESCRIPTIONS OF EMBODIMENTS

Provided herein is a method for determining whether a human subject with breast cancer will respond to therapy with a tyrosine kinase inhibitor or a biologic. The method includes: (a) lysing a breast cancer cell obtained from a sample from the human subject to produce a cellular extract; (b) determining an expression level of truncated HER2 protein, an expression level of full-length HER2 protein, an activation level of full-length HER2 protein, an activation level of HER3 protein, and/or an activation level of PI3K protein in the cellular extract; (c) comparing the expression level of truncated HER2 protein, the expression level of full-length HER2 protein, the activation level of full-length HER2 protein, the activation level of HER3 protein, and/or the activation level of PI3K protein in the cellular extract to a reference expression level of truncated HER2 protein, a reference expression level of full-length HER2 protein, a reference activation level of full-length HER2 protein, a reference activation level of HER3 protein, and/or a reference activation level of PI3K protein, respectively; and (d) determining whether a human subject with breast cancer will respond to therapy with a tyrosine kinase inhibitor or a biologic based upon a difference between the expression level of truncated HER2 protein, the expression level of full-length HER2 protein, the activation level of full-length HER2 protein, the activation level of HER3 protein, and/or the activation level of PI3K protein in the cellular extract compared to the reference expression level of truncated HER2 protein, the reference expression level of full-length HER2 protein, the reference activation level of full-length HER2 protein, the reference activation level of HER3 protein, and/or the reference activation level of PI3K protein, respectively.

In some embodiments, the method for determining whether a human subject with breast cancer will respond to therapy with a tyrosine kinase inhibitor or a biologic includes: (a) determining an expression level of truncated HER2 protein, an expression level of full-length HER2 protein, an activation level of full-length HER2 protein, an activation level of HER3 protein, and/or an activation level of PI3K protein in a cellular extract; (b) comparing the expression level of truncated HER2 protein, the expression level of full-length HER2 protein, the activation level of full-length HER2 protein, the activation level of HER3 protein, and/or the activation level of PI3K protein in the cellular extract to a reference expression level of truncated HER2 protein, a reference expression level of full-length HER2 protein, a reference activation level of full-length HER2 protein, a reference activation level of HER3 protein, and/or a reference activation level of PI3K protein, respectively; and (c) determining whether a human subject with breast cancer will respond to therapy with a tyrosine kinase inhibitor or a biologic based upon a difference between the expression level of truncated HER2 protein, the expression level of full-length HER2 protein, the activation level of full-length HER2 protein, the activation level of HER3 protein, and/or the activation level of PI3K protein in the cellular extract compared to the reference expression level of truncated HER2 protein, the reference expression level of full-length HER2 protein, the reference activation level of full-length HER2 protein, the reference activation level of HER3 protein, and/or the reference activation level of PI3K protein, respectively. In certain instances, the cellular extract is produced by lysing a breast cancer cell obtained from a sample from the human subject.

In some embodiments, the breast cancer is HER2-positive, locally advanced breast cancer.

In some embodiments, the tyrosine kinase inhibitor is a pan-HER inhibitor or a dual HER1/HER2 inhibitor. The pan-HER inhibitor can be selected from the group consisting of neratinib, afatinib, dacomitinib, poziotinib, and combinations thereof. The dual HER1/HER2 inhibitor is selected from the group consisting of lapatinib, AZD8931, BIBW 2992, and combinations thereof. In some instances, the biologic is selected from the group consisting of a monoclonal antibody, an affibody, a probody, a diabody, a dual antibody, a fragment thereof, and combinations thereof. The monoclonal antibody can be an anti-HER2 antibody or an antibody that inhibits HER dimerization. The monoclonal antibody can be an anti-HER2 antibody. In some cases, the anti-HER2 antibody is trastuzumab. The antibody that inhibits HER dimerization can be pertuzumab.

In some embodiments, the therapy is used as neoadjuvant therapy. The neoadjuvant therapy can further comprise paclitaxel, doxorubicin, cyclophosphamide, or combinations thereof.

In one aspect of the present disclosure, the human subject will likely respond to therapy with a tyrosine kinase inhibitor when the expression level of truncated HER2 protein in the cellular extract is higher than the reference expression level of truncated HER2 protein.

In some embodiments, the reference expression level of truncated HER2 protein is a median expression level of truncated HER2 protein in human subjects who did not respond to the tyrosine kinase inhibitor, in human subjects who did not respond to the biologic, and/or in human subjects who responded to the biologic. The expression level of truncated HER2 protein in the cellular extract can be about 3-fold to about 5-fold higher than the median expression level of truncated HER2 protein in subjects who are likely to respond to therapy with the tyrosine kinase inhibitor.

In other embodiments, the expression level of truncated HER2 protein in the cellular extract is a ratio of the expression level of truncated HER2 protein in the cellular extract to an expression level of a control protein. The control protein can be cytokeratin (CK). In some instances, the human subject will likely respond to therapy with the tyrosine kinase inhibitor when the expression level of truncated HER2 protein in the cellular extract is higher than a reference expression level of truncated HER2 protein corresponding to a ratio of about 0.44 relative to the expression level of CK.

In particular embodiments, the present disclosure provides a method for determining whether a human subject with breast cancer will respond to therapy with a tyrosine kinase inhibitor or a biologic and treating the human subject with the tyrosine kinase inhibitor or the biologic, the method comprising: (a) lysing a breast cancer cell obtained from a sample from the human subject to produce a cellular extract; (b) determining an expression level of truncated HER2 protein in the cellular extract; (c) comparing the expression level of truncated HER2 protein in the cellular extract to a reference expression level of truncated HER2 protein as described herein (e.g., comparing to a median expression level or ratio); (d) determining that the human subject will likely be a responder to therapy with a tyrosine kinase inhibitor when the expression level of truncated HER2 protein in the cellular extract is higher than the reference expression level of truncated HER2 protein; and (e) administering an effective amount of a tyrosine kinase inhibitor (e.g., as neoadjuvant therapy) to the responder human subject.

In some embodiments, the method for determining whether a human subject with breast cancer will respond to therapy with a tyrosine kinase inhibitor or a biologic and treating the human subject with the tyrosine kinase inhibitor or the biologic comprises: (a) determining an expression level of truncated HER2 protein in a cellular extract; (b) comparing the expression level of truncated HER2 protein in the cellular extract to a reference expression level of truncated HER2 protein as described herein (e.g., comparing to a median expression level or ratio); (c) determining that the human subject will likely be a responder to therapy with a tyrosine kinase inhibitor when the expression level of truncated HER2 protein in the cellular extract is higher than the reference expression level of truncated HER2 protein; and (d) administering an effective amount of a tyrosine kinase inhibitor (e.g., as neoadjuvant therapy) to the responder human subject. In certain instances, the cellular extract is produced by lysing a breast cancer cell obtained from a sample from the human subject.

In some embodiments, the method further comprises determining an expression level of full-length HER2 protein, an activation level of full-length HER2 protein, an activation level of HER3 protein, and/or an activation level of PI3K protein in the cellular extract.

In another aspect of the present disclosure, the human subject will likely respond to therapy with either a tyrosine kinase inhibitor or a biologic when the expression level of full-length HER2 protein in the cellular extract is higher than the reference expression level of full-length HER2 protein.

In some instances, the reference expression level of full-length HER2 protein is a median expression level of full-length HER2 protein in human subjects who did not respond to the tyrosine kinase inhibitor. In some embodiments, the human subject will likely respond to therapy with the tyrosine kinase inhibitor when the expression level of full-length HER2 protein in the cellular extract is about 2.5-fold higher than the median expression level of full-length HER2 protein.

In other instances, the reference expression level of full-length HER2 protein is a median expression level of full-length HER2 protein in human subjects who did not respond to the biologic. In some embodiments, the human subject will likely respond to therapy with the biologic when the expression level of full-length HER2 protein in the cellular extract is about 2.5-fold higher than the median expression level of full-length HER2 protein.

In some embodiments, the expression level of full-length HER2 protein in the cellular extract is a ratio of the expression level of full-length HER2 protein in the cellular extract to an expression level of a control protein. The control protein can be cytokeratin (CK). In some embodiments, the human subject will likely respond to therapy with the tyrosine kinase inhibitor when the expression level of full-length HER2 protein in the cellular extract is higher than a reference expression level of full-length HER2 protein corresponding to a ratio of about 38.7 relative to the expression level of CK. In other embodiments the human subject will likely respond to therapy with the biologic when the expression level of full-length HER2 protein in the cellular extract is between a reference expression level of full-length HER2 protein corresponding to a ratio of from about 5.6 to about 38.7 relative to the expression level of CK. In some cases, the human subject will likely not respond to therapy with either the tyrosine kinase inhibitor or the biologic when the expression level of full-length HER2 protein in the cellular extract is lower than a reference expression level of full-length HER2 protein corresponding to a ratio of about 5.6 relative to the expression level of CK.

In particular embodiments, the present disclosure provides a method for determining whether a human subject with breast cancer will respond to therapy with a tyrosine kinase inhibitor or a biologic and treating the human subject with the tyrosine kinase inhibitor or the biologic, the method comprising: (a) lysing a breast cancer cell obtained from a sample from the human subject to produce a cellular extract; (b) determining an expression level of full-length HER2 protein in the cellular extract; (c) comparing the expression level of full-length HER2 protein in the cellular extract to a reference expression level of full-length HER2 protein as described herein (e.g., comparing to a median expression level or ratio); (d) determining that the human subject will likely be a responder to therapy with either a tyrosine kinase inhibitor or a biologic when the expression level of full-length HER2 protein in the cellular extract is higher than the reference expression level of full-length HER2 protein; and (e) administering an effective amount of a tyrosine kinase inhibitor and/or a biologic (e.g., as neoadjuvant therapy) to the responder human subject.

In some embodiments, the method for determining whether a human subject with breast cancer will respond to therapy with a tyrosine kinase inhibitor or a biologic and treating the human subject with the tyrosine kinase inhibitor or the biologic comprises: (a) determining an expression level of full-length HER2 protein in a cellular extract; (b) comparing the expression level of full-length HER2 protein in the cellular extract to a reference expression level of full-length HER2 protein as described herein (e.g., comparing to a median expression level or ratio); (c) determining that the human subject will likely be a responder to therapy with either a tyrosine kinase inhibitor or a biologic when the expression level of full-length HER2 protein in the cellular extract is higher than the reference expression level of full-length HER2 protein; and (d) administering an effective amount of a tyrosine kinase inhibitor and/or a biologic (e.g., as neoadjuvant therapy) to the responder human subject. In certain instances, the cellular extract is produced by lysing a breast cancer cell obtained from a sample from the human subject.

In some embodiments, the method further comprises determining an expression level of truncated HER2 protein, an activation level of full-length HER2 protein, an activation level of HER3 protein, and/or an activation level of PI3K protein in the cellular extract.

In yet other aspects of the present disclosure, the human subject will likely respond to therapy with a tyrosine kinase inhibitor when the activation level of full-length HER2 protein in the cellular extract is higher than the reference activation level of full-length HER2 protein. The reference activation level of full-length HER2 protein can be a median activation level of full-length HER2 protein in human subjects who did not respond to the tyrosine kinase inhibitor, in human subjects who did not respond to the biologic, and/or in human subjects who responded to the biologic. In some instances, the human subject will likely respond to therapy with the tyrosine kinase inhibitor when the activation level of full-length HER2 protein in the cellular extract is about 3-fold to about 7-fold higher than the median activation level of full-length HER2 protein.

In some embodiments, the activation level of full-length HER2 protein in the cellular extract is a ratio of the activation level of full-length HER2 protein in the cellular extract to an expression level of a control protein. The control protein can be cytokeratin (CK). In some cases, the human subject will likely respond to therapy with the tyrosine kinase inhibitor when the activation level of full-length HER2 protein in the cellular extract is higher than a reference activation level of full-length HER2 protein corresponding to a ratio of about 3.1 relative to the expression level of CK. In other cases, the human subject will likely not respond to therapy with either the tyrosine kinase inhibitor or the biologic when the activation level of full-length HER2 protein in the cellular extract is lower than a reference activation level of full-length HER2 protein corresponding to a ratio of about 0.3 relative to the expression level of CK.

In particular embodiments, the present disclosure provides a method for determining whether a human subject with breast cancer will respond to therapy with a tyrosine kinase inhibitor or a biologic and treating the human subject with the tyrosine kinase inhibitor or the biologic, the method comprising: (a) lysing a breast cancer cell obtained from a sample from the human subject to produce a cellular extract; (b) determining an activation level of full-length HER2 protein in the cellular extract; (c) comparing the activation level of full-length HER2 protein in the cellular extract to a reference activation level of full-length HER2 protein as described herein (e.g., comparing to a median activation level or ratio); (d) determining that the human subject will likely be a responder to therapy with a tyrosine kinase inhibitor when the activation level of full-length HER2 protein in the cellular extract is higher than the reference activation level of full-length HER2 protein; and (e) administering an effective amount of a tyrosine kinase inhibitor (e.g., as neoadjuvant therapy) to the responder human subject.

In some embodiments, the method for determining whether a human subject with breast cancer will respond to therapy with a tyrosine kinase inhibitor or a biologic and treating the human subject with the tyrosine kinase inhibitor or the biologic comprises: (a) determining an activation level of full-length HER2 protein in a cellular extract; (b) comparing the activation level of full-length HER2 protein in the cellular extract to a reference activation level of full-length HER2 protein as described herein (e.g., comparing to a median activation level or ratio); (c) determining that the human subject will likely be a responder to therapy with a tyrosine kinase inhibitor when the activation level of full-length HER2 protein in the cellular extract is higher than the reference activation level of full-length HER2 protein; and (d) administering an effective amount of a tyrosine kinase inhibitor (e.g., as neoadjuvant therapy) to the responder human subject. In certain instances, the cellular extract is produced by lysing a breast cancer cell obtained from a sample from the human subject.

In some embodiments, the method further comprises determining an expression level of truncated HER2 protein, an expression level of full-length HER2 protein, an activation level of HER3 protein, and/or an activation level of PI3K protein in the cellular extract.

In some aspects of the present disclosure, the human subject will likely respond to therapy with a tyrosine kinase inhibitor when the activation level of HER3 protein in the cellular extract is higher than the reference activation level of HER3 protein. The reference activation level of HER3 protein can be a median activation level of HER3 protein in human subjects who did not respond to the tyrosine kinase inhibitor, in human subjects who did not respond to the biologic, and/or in human subjects who responded to the biologic. In some cases, the human subject will likely respond to therapy with a tyrosine kinase inhibitor when the activation level of HER3 protein in the cellular extract is about 1-fold to about 3.5-fold higher than the median activation level of HER3 protein.

In other aspects of the present disclosure, the human subject will likely respond to therapy with a biologic when the activation level of HER3 protein in the cellular extract is lower than the reference activation level of HER3 protein. The reference activation level of HER3 protein can be a median activation level of HER3 protein in human subjects who did not respond to the biologic, in human subjects who did not respond to the tyrosine kinase inhibitor, and/or in human subjects who responded to the tyrosine kinase inhibitor. In some instances, the activation level of HER3 protein in the cellular extract is about 1-fold to about 3.5-fold lower than the median activation level of HER3 protein.

In some cases, the activation level of HER3 protein in the cellular extract can be a ratio of the activation level of HER3 protein in the cellular extract to an expression level of a control protein. In some cases, the control protein is cytokeratin (CK). In some embodiments, the human subject will likely respond to therapy with the tyrosine kinase inhibitor when the activation level of HER3 protein in the cellular extract is higher than a reference activation level of HER3 protein corresponding to a ratio of about 0.2 relative to the expression level of CK. In other embodiments, the human subject will likely respond to therapy with the biologic when the activation level of HER3 protein in the cellular extract is lower than a reference activation level of HER3 protein corresponding to a ratio of about 0.2 relative to the expression level of CK.

In particular embodiments, the present disclosure provides a method for determining whether a human subject with breast cancer will respond to therapy with a tyrosine kinase inhibitor or a biologic and treating the human subject with the tyrosine kinase inhibitor or the biologic, the method comprising: (a) lysing a breast cancer cell obtained from a sample from the human subject to produce a cellular extract; (b) determining an activation level of HER3 protein in the cellular extract; (c) comparing the activation level of HER3 protein in the cellular extract to a reference activation level of HER3 protein as described herein (e.g., comparing to a median activation level or ratio); (d) determining that the human subject will likely be a responder to therapy with a tyrosine kinase inhibitor when the activation level of HER3 protein in the cellular extract is higher than the reference activation level of HER3 protein, or determining that the human subject will likely be a responder to therapy with a biologic when the activation level of HER3 protein in the cellular extract is lower than the reference activation level of HER3 protein; and (e) administering an effective amount of a tyrosine kinase inhibitor or a biologic (e.g., as neoadjuvant therapy) to the responder human subject.

In some embodiments, the method for determining whether a human subject with breast cancer will respond to therapy with a tyrosine kinase inhibitor or a biologic and treating the human subject with the tyrosine kinase inhibitor or the biologic comprises: (a) determining an activation level of HER3 protein in a cellular extract; (b) comparing the activation level of HER3 protein in the cellular extract to a reference activation level of HER3 protein as described herein (e.g., comparing to a median activation level or ratio); (c) determining that the human subject will likely be a responder to therapy with a tyrosine kinase inhibitor when the activation level of HER3 protein in the cellular extract is higher than the reference activation level of HER3 protein, or determining that the human subject will likely be a responder to therapy with a biologic when the activation level of HER3 protein in the cellular extract is lower than the reference activation level of HER3 protein; and (d) administering an effective amount of a tyrosine kinase inhibitor or a biologic (e.g., as neoadjuvant therapy) to the responder human subject. In certain instances, the cellular extract is produced by lysing a breast cancer cell obtained from a sample from the human subject.

In some embodiments, the method further comprises determining an expression level of truncated HER2 protein, an expression level of full-length HER2 protein, an activation level of full-length HER2 protein, and/or an activation level of PI3K protein in the cellular extract.

In some aspects of the present disclosure, the human subject will likely respond to therapy with a tyrosine kinase inhibitor when the activation level of PI3K protein in the cellular extract is higher than the reference activation level of PI3K protein. The reference activation level of full-length PI3K protein can be a median activation level of PI3K protein in human subjects who did not respond to the tyrosine kinase inhibitor, in human subjects who did not respond to the biologic, and/or in human subjects who responded to the biologic. In some instances, the activation level of PI3K protein in the cellular extract is about 0.5-fold to about 3.5-fold higher than the median activation level of PI3K protein.

In other aspects of the present disclosure, the human subject will likely respond to therapy with a biologic when the activation level of PI3K protein in the cellular extract is lower than the reference activation level of PI3K protein. The reference activation level of full-length PI3K protein can be a median activation level of PI3K protein in human subjects who did not respond to the biologic, in human subjects who did not respond to the tyrosine kinase inhibitor, and/or in human subjects who responded to the tyrosine kinase inhibitor. In some cases, the activation level of PI3K protein in the cellular extract is about 0.5-fold to about 3.5-fold lower than the median activation level of PI3K protein.

In some embodiments, the activation level of PI3K protein in the cellular extract can be a ratio of the activation level of PI3K protein in the cellular extract to an expression level of a control protein. The control protein can be cytokeratin (CK). In some cases, the human subject will likely respond to therapy with the tyrosine kinase inhibitor when the activation level of PI3K protein in the cellular extract is higher than a reference activation level of PI3K protein corresponding to a ratio of about 0.04 relative to the expression level of CK.

In particular embodiments, the present disclosure provides a method for determining whether a human subject with breast cancer will respond to therapy with a tyrosine kinase inhibitor or a biologic and treating the human subject with the tyrosine kinase inhibitor or the biologic, the method comprising: (a) lysing a breast cancer cell obtained from a sample from the human subject to produce a cellular extract; (b) determining an activation level of PI3K protein in the cellular extract; (c) comparing the activation level of PI3K protein in the cellular extract to a reference activation level of PI3K protein as described herein (e.g., comparing to a median activation level or ratio); (d) determining that the human subject will likely be a responder to therapy with a tyrosine kinase inhibitor when the activation level of PI3K protein in the cellular extract is higher than the reference activation level of PI3K protein, or the human subject will likely be a responder to therapy with a biologic when the activation level of PI3K protein in the cellular extract is lower than the reference activation level of PI3K protein; and (e) administering an effective amount of a tyrosine kinase inhibitor or a biologic (e.g., as neoadjuvant therapy) to the responder human subject.

In some embodiments, the method for determining whether a human subject with breast cancer will respond to therapy with a tyrosine kinase inhibitor or a biologic and treating the human subject with the tyrosine kinase inhibitor or the biologic comprises: (a) determining an activation level of PI3K protein in a cellular extract; (b) comparing the activation level of PI3K protein in the cellular extract to a reference activation level of PI3K protein as described herein (e.g., comparing to a median activation level or ratio); (c) determining that the human subject will likely be a responder to therapy with a tyrosine kinase inhibitor when the activation level of PI3K protein in the cellular extract is higher than the reference activation level of PI3K protein, or the human subject will likely be a responder to therapy with a biologic when the activation level of PI3K protein in the cellular extract is lower than the reference activation level of PI3K protein; and (d) administering an effective amount of a tyrosine kinase inhibitor or a biologic (e.g., as neoadjuvant therapy) to the responder human subject. In certain instances, the cellular extract is produced by lysing a breast cancer cell obtained from a sample from the human subject.

In some embodiments, the method further comprises determining an expression level of truncated HER2 protein, an expression level of full-length HER2 protein, an activation level of full-length HER2 protein, and/or an activation level of HER3 protein in the cellular extract.

In the method provided herein, the expression level of truncated HER2 protein, the expression level of full-length HER2 protein, and the activation level of full-length HER2 protein can be determined. Alternatively, the expression level of full-length HER2 protein and the activation level of HER3 protein is determined.

In some embodiments, the method further comprises determining an expression level and/or an activation level of one or more additional signal transduction molecules in the cellular extract. The one or more additional signal transduction molecules can be selected from the group consisting of AKT, PRAS40, ERK1 (MAPK3), ERK2 (MAPK1), RSK, and combinations thereof.

In some embodiments, the sample is a breast tumor tissue, whole blood, serum, or plasma sample. The breast tumor tissue sample can be a needle biopsy sample. In certain embodiments, the method further comprises obtaining the sample from the human subject.

The expression level of truncated HER2 protein, the expression level of full-length HER2 protein, the activation level of full-length HER2 protein, the activation level of full-length HER3 protein, and/or the activation level of full-length PI3K protein can be determined with Collaborative Enzyme Enhanced Reactive ImmunoAssay (CEER). The method can further comprise administering the tyrosine kinase inhibitor or the biologic to the human subject.

A. Quantitating Expression Levels and/or Activation Level of Analytes in Cellular Extracts

The subject of the present invention can be a human subject with breast cancer. The type of breast cancer can be ductal carcinoma in situ, invasive ductal carcinoma, triple negative breast cancer, inflammatory breast cancer (locally advanced breast cancer), metastatic cancer, medullary carcinoma, tubular carcinoma, mucinous carcinoma, mammary Paget disease, or other types of breast cancer. In some instances, the subject has stage 0 (non-invasive breast cancer), stage I (early or non-invasive breast cancer), stage I or II (early or non-invasive breast cancer), stage II or III (locally advanced breast cancer), or stage IV (advanced breast cancer) breast cancer. In some embodiments, the human subject has HER2-positive breast cancer. Such subjects have breast cancer cells that carry a HER2 gene mutation that results in overexpression of HER2 protein.

A breast cancer cell can be obtained from a sample taken from the subject. The breast cancer cell can be isolated from the sample using one or more separation methods including, for example, immunomagnetic separation (see, e.g., Racila et al., Proc. Natl. Acad. Sci. USA, 95:4589-4594 (1998); Bilkenroth et al., Int. J. Cancer, 92:577-582 (2001)), microfluidic separation (see, e.g., Mohamed et al., IEEE Trans. Nanobiosci., 3:251-256 (2004); Lin et al., Abstract No. 5147, 97th AACR Annual Meeting, Washington, D.C. (2006)), FACS (see, e.g., Mancuso et al., Blood, 97:3658-3661 (2001)), density gradient centrifugation (see, e.g., Baker et al., Clin. Cancer Res., 13:4865-4871 (2003)), and depletion methods (see, e.g., Meye et al., Int. J. Oncol., 21:521-530 (2002)). The breast cancer cell can be a circulating tumor cell. In some embodiments, the sample obtained from the subject is a breast tumor tissue sample, a blood sample, a serum sample, or a plasma sample. In other cases, the breast cancer cell can be obtained from a needle biopsy of a tumor. The isolated breast cancer cell can be lysed to produce a cellular extract using any method known to one of ordinary skill in the art.

The expression level and/or activation level of one or more analytes of the HER1 signaling pathway, HER2 signaling pathway, HER3 signaling pathway, or other signaling pathways associated with cancer can be detected or measured. In some embodiments, the expression level of truncated HER2 protein (e.g., p95HER2 protein), the expression level of full-length HER2 protein, the expression level of HER3 protein, the expression level of PI3K protein, the expression level of ERK1 (MAPK3) protein, the expression level of ERK2 (MAPK1) protein, the expression level of RSK protein, the expression level of AKT protein, the expression level of PRAS40 protein, and any combination thereof are determined. In other embodiments, the activation level of truncated HER2 protein (e.g., p95HER2 protein), the activation level of full-length HER2 protein, the activation level of HER3 protein, the activation level of PI3K protein, the activation level of ERK1 (MAPK3) protein, the activation level of ERK2 (MAPK1) protein, the activation level of RSK protein, the activation level of AKT protein, the activation level of PRAS40 protein, and any combination thereof are determined. The expression level of 1, 2, 3, 4, 5, 6, 7, 8, 9, or more analytes (signal transduction molecules) described herein can be quantitated. In some cases, the activation level of 1, 2, 3, 4, 5, 6, 7, 8, 9, or more analytes (signal transduction molecules) described herein are quantitated. In other cases, the expression level of 1, 2, 3, 4, 5, 6, 7, 8, 9, or more analytes, and the activation level of 1, 2, 3, 4, 5, 6, 7, 8, 9, or more of the same or different analytes are measured.

Methods of detecting expression and/or activation level of any one or the analytes described herein include immunoassays (e.g., enzyme-linked immunosorbent assays), mass spectrometry, flow cytometry, immunocytochemistry, immunohistochemistry, Western blotting, kinase activity assays, and other protein activity assays. In some embodiments, the expression and/or activation level is determined using a Collaborative Enzyme Enhanced Reactive Immunoassay (CEER™). Descriptions of exemplary CEER™ assays are found in, for example, U.S. Pat. Nos. 8,609,349; 8,658,388; 9,250,243; 9,274,116; and 9,285,369; Lim et al., Proteome Sci, 2011, 9:75; and Lee et al., PLoS One, 2013, 8(1): e54644.

The expression level of truncated HER2 protein, the expression level of full-length HER2 protein, the activation level of full-length HER2 protein, the expression level of HER3 protein, the activation level of HER3 protein, the expression level of PI3K protein and/or the activation level of PI3K protein is detected using CEER™. In some embodiments, the expression level and/or activation level of AKT, PRAS40, ERK1, ERK2, RSK and any combination thereof can also be determined using such an assay.

B. Predicting Likely Responders or Non-Responders to Therapy Containing a Tyrosine Kinase Inhibitor or a Biologic

To determine if the subject is likely to respond (e.g., likely to have a clinical response) to a drug therapy comprising a tyrosine kinase inhibitor (TKI) or a biologic, the expression level of an analyte(s), such as truncated HER2 protein (p95HER2 protein), full-length HER2 protein, HER3 protein, PI3K protein, ERK1 (MAPK3) protein, ERK2 (MAPK1) protein, RSK protein, AKT protein, PRAS40 protein, and RPS6 protein can be compared to a reference expression level of the corresponding protein. For example, the reference expression level of p95HER2 protein can be compared to the expression level of p95HER2 protein in the cellular extract derived from the subject's sample. In some embodiments, a prediction of response to a drug therapy is based on comparing the activation level (e.g., phosphorylation level) of an analyte(s), such as full-length HER1 protein, truncated HER2 protein (p95HER2 protein), full-length HER2 protein, HER3 protein, PI3K protein, ERK1 (MAPK3) protein, ERK2 (MAPK1) protein, RSK protein, AKT protein, PRAS40 protein, RPS6 protein to a reference activation level of the corresponding protein. For instances, the reference activation level of HER2 protein (e.g., the reference level of activated HER2 protein) can be compared to the activation level of HER2 protein in the cellular extract derived from the subject's sample. A comparison can be made between: the determined expression level of truncated HER2 protein and a reference expression level of truncated HER2 protein; the determined expression level of full-length HER2 protein and a reference expression level of full-length HER2 protein; the determined expression level of HER3 protein and a reference expression level of HER3 protein; the determined expression level of PI3K protein and a reference expression level of PI3K protein; the determined expression level of ERK1 protein and a reference expression level of ERK1 protein; the determined expression level of ERK2 protein and a reference expression level of ERK2 protein the determined expression level of RSK protein and a reference expression level of RSK protein; the determined expression level of RPS6 protein and a reference expression level of RPS6 protein; the determined expression level of AKT protein and a reference expression level of AKT protein; the determined expression level of PRAS40 protein and a reference expression level of PRAS40 protein; the determined activation level of truncated HER2 protein and a reference activation level of truncated HER2 protein; the determined activation level of full-length HER2 protein and a reference activation level of full-length HER2 protein; the determined activation level of HER3 protein and a reference activation level of HER3 protein; the determined activation level of PI3K protein and a reference activation level of PI3K protein; the determined activation level of ERK1 protein and a reference activation level of ERK1 protein; the determined activation level of ERK2 protein and a reference activation level of ERK2 protein; the determined activation level of RSK protein and a reference activation level of RSK protein; the determined activation level of RPS6 protein and a reference activation level of RPS6 protein; the determined activation level of AKT protein and a reference activation level of AKT protein; and/or the determined activation level of PRAS40 protein and a reference activation level of PRAS40 protein.

In some embodiments, a subject will likely respond to a drug therapy comprising a tyrosine kinase inhibitor or a biologic when the subject has greater than a 50% probability of responding to the therapy, e.g., at least a 50%, 55%, 60%, 65%, 70%, 75%, 80%, 85%, 90%, or 95% probability of responding to the therapy. In other embodiments, a subject will likely not respond to a drug therapy comprising a tyrosine kinase inhibitor or a biologic when the subject has less than a 50% probability of responding to the therapy, e.g., less than a 50%, 45%, 40%, 35%, 30%, 25%, 20%, 15%, 10%, or 5% probability of responding to the therapy. In certain embodiments, a response to therapy corresponds to achieving a clinical response such as a pathological complete response in breast and nodes (pCRBN).

A subject is predicted to likely respond to a therapy comprising a tyrosine kinase inhibitor if the expression level of truncated HER2 protein (p95HER2 protein) in the cellular extract produced from the subject's sample is higher than the reference expression level of p95HER2 protein. In some cases, the reference expression level is the median expression level of p95HER2 protein in one or more populations of subject including human subjects who received a therapy comprising a tyrosine kinase inhibitor and did not have a positive or clinical response to the therapy, human subjects who received a therapy comprising a biologic and did not have a positive response or clinical response to the therapy, and human subjects who received a therapy comprising a biologic and had a positive response or clinical response to the therapy.

In some instances, the higher expression level of p95HER2 protein can be about 3-fold to about 5-fold higher, e.g., about 3-fold higher, about 3.5-fold higher, about 4-fold higher, about 4.5-fold higher, or about 5-fold higher, than the median expression level of truncated HER2 protein. In other instances, the higher expression level of p95HER2 is about 1-fold to about 10-fold higher, e.g., about 1-fold higher, about 1.5-fold higher, about 2-fold higher, about 2.5-fold higher, about 3-fold higher, 3.5-fold higher, about 4-fold higher, about 4.5-fold higher, about 5-fold higher, about 5.5-fold higher, about 6-fold higher, about 6.5-fold higher, about 7-fold higher, 7.5-fold higher, about 8-fold higher, about 8.5-fold higher, about 9-fold higher, 9.5-fold higher, or about 10-fold higher, than the median expression level of p95HER2 protein.

The expression level of truncated HER2 protein in the cellular extract produced from the subject's sample can be represented as a ratio of the expression level of truncated HER2 protein in this cellular extract relative to an expression level of a control protein. The control protein can be cytokeratin, or any protein that can serve as an assay control protein. In some instances, a subject is likely to respond to a therapy comprising a tyrosine kinase inhibitor if the ratio of the expression level of p95HER2 to cytokeratin is higher than a reference ratio of the expression level of p95HER2 to cytokeratin. In some cases, the reference ratio is about 0.44. As such, a subject is predicted to be a responder to tyrosine kinase inhibitor therapy if the subject's expression level of p95HER2 protein (as represented as a ratio of p95HER2 expression to CK expression) is higher than 0.44, e.g., 0.45, 0.50, 0.55, 0.60, 0.65, 0.70, 0.8, 0.9, or more.

A subject is predicted to likely respond to a therapy comprising a tyrosine kinase inhibitor or a biologic if the expression level of full-length HER2 protein in the cellular extract produced from the subject's sample is higher than the reference expression level of full-length HER2 protein.

In some embodiments, the reference expression level of full-length HER2 is a median expression level of full-length HER2 protein in a population of human subject who were administered tyrosine kinase inhibitor therapy and did not respond to it. In some instances, a subject is likely to respond to a therapy comprising a tyrosine kinase inhibitor when the determined expression level of full-length HER2 protein is about 2.5-fold higher or more, e.g., about 2.5-fold higher, about 3.0-fold higher, about 3.5-fold higher, about 4.0-fold higher, about 4.5-fold higher, about 5.0-fold hire, or more than the median expression level of full-length HER2 protein in a population of human subjects who were administered tyrosine kinase inhibitor therapy and did not respond to it. In other instances, the higher expression level of full-length HER2 protein is about 2.0-fold higher to about 5.0-fold higher, e.g., about 2.0-fold higher to about 5.0-fold higher, about 3.0-fold higher to about 5.0-fold higher, about 2.0-fold higher to about 4.0-fold higher, about 2.0-fold higher to about 3.0-fold higher, about 3.0-fold higher to about 4.0-fold higher, about 4.0-fold higher to about 5.0-fold higher, about 2.0-fold higher, about 2.5-fold higher, about 3.0-fold higher, about 3.5-fold higher, about 4.0-fold higher, about 4.5-fold higher, or about 5.0-fold higher than the median expression of full-length HER2 protein in human subject who were administered tyrosine kinase inhibitor therapy and did not respond to it.

In other embodiments, the reference expression level of full-length HER2 is a median expression level of full-length HER2 protein in a population of human subject who was administered a therapy comprising a biologic and did not respond to it. In some instances, a subject is likely to respond to a therapy comprising a TKI when the determined expression level of full-length HER2 protein is about 2.5-fold higher or more, e.g., about 2.5-fold, about 3.0-fold, about 3.5-fold, about 4.0-fold, about 4.5-fold, about 5.0-fold or more higher than the median expression level of full-length HER2 protein in a population of human subjects who were administered biologic therapy and did not respond to it. In other instances, a subject is likely to respond to a therapy comprising a TKI if the subject's expression level of full-length HER2 protein is about 2.0-fold higher to about 5.0-fold higher, e.g., about 2.0-fold higher to about 5.0-fold higher, about 3.0-fold higher to about 5.0-fold higher, about 2.0-fold higher to about 4.0-fold higher, about 2.0-fold higher to about 3.0-fold higher, about 3.0-fold higher to about 4.0-fold higher, about 4.0-fold higher to about 5.0-fold higher, about 2.0-fold higher, about 2.5-fold higher, about 3.0-fold higher, about 3.5-fold higher, about 4.0-fold higher, about 4.5-fold higher, or about 5.0-fold higher than the median expression level of full-length HER2 protein in a population of human subjects who were administered biologic therapy and did not respond to it.

In some embodiments, the expression level of full-length HER2 protein in the cellular extract produced from the subject's sample is a ratio of the expression level of full-length HER2 protein in this cellular extract to an expression level of a control protein. The control protein can be cytokeratin (CK), or any protein that can serve as an assay control protein. In some instances, a subject is likely to respond to a therapy comprising a TKI if the ratio of the expression level of full-length HER2 protein to the expression level of cytokeratin protein is higher than about 38.7. For example, a subject having an expression level of full-length HER2 (as represented by a ratio of HER2 expression level to CK expression level) of higher than about 38.7, e.g., about 38.8, about 39, about 40, about 41, about 42, about 43, about 44, about 45, about 46, about 47, about 48, about 49, about 50, about 55, about 60, about 65 or more is predicted to respond to a tyrosine kinase inhibitor.

In other instances, a subject is likely to respond to a therapy comprising a biologic when the subject's ratio of expression level of full-length HER2 protein to expression level of cytokeratin protein is from about 5.6 to about 38.7, e.g., about 5.6 to about 38.7, about 8.0 to about 38.0, about 10.0 to about 30.0, about 15.0 to about 30.0, about 20.0 to about 30.0, about 25.0 to about 38.7, about 5.6 to about 10.0, about 5.6, about 10.0, about 15.6, about 20.0, about 25.6, about 30.0, about 35.6, about 38.7, and the like.

In yet other instances, a subject is likely to not respond to therapy containing either a TKI or a biologic when the subject's ratio of expression level of full-length HER2 protein to expression level of cytokeratin protein is lower than about 5.6, e.g., about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, or lower.

A subject can be predicted to likely respond to a therapy comprising a tyrosine kinase inhibitor if the activation level of full-length HER2 protein in the cellular extract produced from the subject's sample is higher than the reference activation level of full-length HER2 protein. The reference activation level of HER2 protein can be a median activation level of HER2 protein in a population of human subjects who received a therapy comprising a tyrosine kinase inhibitor and did not respond to it. Alternatively, the reference activation level of HER2 protein can be a median activation level of HER2 protein in a population of human subjects who received a therapy comprising a biologic and responded to it. In other cases, the reference activation level of HER2 protein can be a median activation level of HER2 protein in a population of human subjects who received a therapy comprising a biologic and failed to respond to it. In some embodiments, if the activation level of HER2 protein in a subject's cellular extract is higher than the median activation level of full-length HER2 protein in a population of human subject who received a therapy comprising a tyrosine kinase inhibitor and did not have a positive or clinical response to the therapy, then the subject is predicted to respond to tyrosine kinase inhibitor therapy. In other embodiments, the subject is likely to respond to a tyrosine kinase inhibitor if the activation level of HER2 protein is higher than the median activation level of HER2 protein in a population of human subjects who received a therapy comprising a biologic and did not have a positive response or clinical response to the therapy. In yet other embodiments, the subject is likely to respond to a tyrosine kinase inhibitor, if the activation level of HER2 protein is higher than the median activation level of HER2 protein in a population of human subjects who received a therapy comprising a biologic and had a positive response or clinical response to the therapy.

The higher activation level of HER2 can be about 3-fold to about 7-fold, e.g., about 3-fold to about 7-fold, about 3-fold to about 6-fold, about 3-fold to about 5-fold, about 4-fold to about 7-fold, about 5-fold to about 7-fold, about 3-fold to about 4-fold, about 4-fold to about 5-fold, about 5-fold to about 6-fold, or about 6-fold to about 7-fold higher than the median HER2 activation level. In other cases, the higher (elevated or increased) activation level of HER2 protein is about 3-fold to about 7-fold higher, e.g., about 3-fold higher, about 3.5-fold higher, about 4-fold higher, about 4.5-fold higher, about 5-fold higher, about 5.5-fold higher, about 6-fold higher, about 6.5-fold higher, or about 7-fold higher the median HER2 activation level.

In some embodiments, the activation level of full-length HER2 protein in the cellular extract produced from the subject's sample is a ratio of the activation level of full-length HER2 protein in this cellular extract to an expression level of a control protein. The control protein can be cytokeratin, or any protein that can serve as an assay control protein. In some instances, a subject is likely to respond to a therapy comprising a TKI if the subject's ratio of the activation level of HER2 to the expression level of cytokeratin is higher than a reference ratio (corresponding to the reference activation level of HER2 to the reference expression level of cytokeratin) of 3.1. In other words, the subject is likely to be a responder to a TKI, when the subject's ratio of activation level of full-length HER2 protein to expression level of cytokeratin protein is higher than about 3.1, e.g., about 3.2, about 3.4, about 3.6, about 3.8, about 4.0, about 4.2, about 4.4, about 4.6, about 4.8, about 5.0, about 5.2, about 5.4, about 5.6, about 5.8, about 6.0, about 6.2, about 6.4, about 6.6, about 6.8, about 7.0, about 7.2, about 7.4, about 7.6, or higher. In other instances, a subject is likely to not respond to a therapy of either a TKI or a biologic if the subject's ratio of the activation level of HER2 to the expression level of cytokeratin is lower than a reference ratio of about 0.3. This subject can have an activation level of full-length HER2 protein (corresponding to a ratio) that is lower than about 0.3, e.g., about 0.29, about 0.28, about 0.27, about 0.26, about 0.25, about 0.24, about 0.23, about 0.22, about 0.21, about 0.20, about 0.19, about 0.18, about 0.17, about 0.16, about 0.15, or less.

A subject can be predicted to likely respond to a therapy comprising a tyrosine kinase inhibitor if the activation level of HER3 protein in the cellular extract produced from the subject's sample is higher than the reference activation level of HER3 protein. The reference activation level of HER3 protein can be a median activation level of HER3 protein in a population of human subjects who received a therapy comprising a tyrosine kinase inhibitor and did not respond to it. Alternatively, the reference activation level of HER3 protein can be a median activation level of HER3 protein in a population of human subjects who received a therapy comprising a biologic and responded to it. In other cases, the reference activation level of HER3 protein can be a median activation level of HER3 protein in a population of human subjects who received a therapy comprising a biologic and failed to respond to it.

In some embodiments, a higher activation level of HER3 protein compared to the median activation level is about 1-fold to about 3.5-fold higher, e.g., about 1-fold to about 3.5-fold higher, about 1-fold to about 3-fold higher, about 1-fold to about 2-fold higher, about 2-fold to about 3.5-fold higher, or about 3-fold to about 3.5-fold higher than the median HER3 activation level. In other cases, the higher (elevated or increased) activation level of HER3 protein is about 1-fold to about 3.5-fold higher, e.g., about 1-fold higher, about 1.5-fold higher, about 2-fold higher, about 2.5-fold higher, about 3-fold higher, or about 3.5-fold higher than the median HER3 activation level.

A subject can be predicted to likely respond to a therapy comprising a biologic if the activation level of HER3 protein in the cellular extract produced from the subject's sample is lower than the reference activation level of HER3 protein. The reference activation level of HER3 protein can be a median activation level of HER3 protein in a population of human subjects who received a therapy comprising a biologic and did not respond to it. Alternatively, the reference activation level of HER3 protein can be a median activation level of HER3 protein in a population of human subjects who received a therapy comprising a tyrosine kinase inhibitor and responded to it. In other cases, the reference activation level of HER3 protein can be a median activation level of HER3 protein in a population of human subjects who received a therapy comprising a tyrosine kinase inhibitor and failed to respond.

In some embodiments, a lower activation level of HER3 protein compared to the median activation level is about 1-fold to about 3.5-fold lower, e.g., about 1-fold to about 3.5-fold lower, about 1-fold to about 3-fold lower, about 1-fold to about 2-fold lower, about 2-fold to about 3.5-fold lower, or about 3-fold to about 3.5-fold lower than the median HER3 activation level. In some cases, the lower (reduced or decreased) activation level of HER3 protein is about 1-fold to about 3.5-fold lower, e.g., about 1-fold lower, about 1.5-fold lower, about 2-fold higher, about 2.5-fold lower, about 3-fold lower, or about 3.5-fold lower than the median HER3 activation level.

In some embodiments, the activation level of HER3 protein in the cellular extract produced from the subject's sample is a ratio of the activation level of HER3 protein in this cellular extract to an expression level of a control protein. The control protein can be cytokeratin, or any protein that can serve as an assay control protein. In some instances, a subject is likely to respond to a therapy comprising a TKI if the subject's ratio of the activation level of HER3 to the expression level of cytokeratin is higher than 0.2 (or the reference ratio of the reference activation level of HER3 protein relative to the reference expression level of cytokeratin. A responder to the TKI can have a ratio of activated HER3 protein to expression of cytokeratin protein that is higher than about 0.2, e.g., about 0.25, about 0.3, about 0.4, about 0.5, about 0.6, about 0.7, about 0.8, about 0.9, about 1.0, about 1.1, about 1.2 or more. In other instances, a subject is likely to respond to a therapy comprising a biologic if the ratio of the activation level of HER3 to the expression level of cytokeratin is lower than 0.2 (or the reference ratio of the reference activation level of HER3 protein to the reference expression level of cytokeratin. A responder to the biologic can have a ratio of activated HER3 protein to expression of cytokeratin protein that is less than about 0.2, e.g., about 0.19, about 0.18, about 0.17, about 0.16, about 0.15, about 0.14, about 0.13, about 0.12, about 0.11, about 0.10, about 0.09, about 0.08, about 0.07, about 0.06, about 0.05 or less.

A subject can be predicted to likely respond to a therapy comprising a TKI if the activation level of PI3K protein in the cellular extract produced from the subject's sample is higher than the reference activation level of PI3K protein. The reference activation level of PI3K protein can be a median activation level of PI3K protein in a population of human subjects who received a therapy comprising a TKI and did not respond to it. Alternatively, the reference activation level of PI3K protein can be a median activation level of PI3K protein in a population of human subjects who received a therapy comprising a biologic and responded to it. In other cases, the reference activation level of PI3K protein can be a median activation level of PI3K protein in a population of human subjects who received a therapy comprising a biologic and failed to respond to it.

In some embodiments, a higher activation level of PI3K protein compared to the median activation level is about 0.5-fold to about 3.5-fold higher, e.g., about 0.5-fold to about 3.5-fold higher, about 0.5-fold to about 3-fold higher, about 0.5-fold to about 2-fold higher, about 0.5-fold to about 1.5-fold higher, about 0.5-fold to about 1-fold higher, about 1-fold to about 3.5-fold higher, about 2-fold to about 3.5-fold higher, about 3-fold to about 3.5-fold higher, about 1-fold to about 2-fold higher, or about 2-fold to about 3-fold higher than the median PI3K activation level. In other cases, the higher (elevated or increased) activation level of PI3K protein is about 0.5-fold to about 3.5-fold higher, e.g., about 0.5-fold higher, about 1-fold higher, about 1.5-fold higher, about 2-fold higher, about 2.5-fold higher, about 3-fold higher, or about 3.5-fold higher than the median PI3K activation level.

A subject can be predicted to likely respond to a therapy comprising a biologic if the activation level of PI3K protein in the cellular extract produced from the subject's sample is lower than the reference activation level of PI3K protein. The reference activation level of PI3K protein can be a median activation level of PI3K protein in a population of human subjects who received a therapy comprising a biologic and did not respond to it. Alternatively, the reference activation level of PI3K protein can be a median activation level of PI3K protein in a population of human subjects who received a therapy comprising a TKI and responded to it. In other cases, the reference activation level of PI3K protein can be a median activation level of PI3K protein in a population of human subjects who received a therapy comprising a TKI and failed to respond to it.

In some cases, if the activated PI3K level in the subject's cellular extract is about 0.5-fold to about 3.5-fold lower, e.g., about 0.5-fold to about 3.5-fold lower, about 1.0-fold to about 3.5-fold lower, about 1.5-fold to about 3.5-fold lower, about 2.0-fold to about 3.5-fold lower, about 2.5-fold to about 3.5-fold lower, 0.5-fold to about 3.0-fold lower, 0.5-fold to 2.5-fold lower, 0.5-fold to about 2.0-fold lower, 0.5-fold to about 1.5-fold lower, 0.5-fold to about 1.0-fold lower, 0.5-fold lower, 1.0-fold lower, 1.5-fold lower, 2.0-fold lower, 2.5-fold lower, 3.0-fold lower, or 3.5-fold lower than the median activated PI3K level, then the subject is likely to respond to a biologic.

In some embodiments, the activation level of PI3K protein in the cellular extract produced from the subject's sample is a ratio of the activation level of PI3K protein to an expression level of a control protein. The control protein can be cytokeratin, or any protein that can serve as an assay control protein. In some instances, a subject is likely to respond to a therapy comprising a TKI if the subject's ratio of the activation level of PI3K to the expression level of cytokeratin is higher than 0.04 (or the reference ratio of the reference activation level of PI3K protein relative to the reference expression level of cytokeratin. A responder to the TKI can have a ratio of activated PI3K protein to expression of cytokeratin protein that is higher than about 0.04, e.g., about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, about 0.30, about 0.40, about 0.50, about 0.60, or more.

In other instances, a subject is likely to respond to a therapy comprising a biologic if the ratio of the activation level of PI3K to the expression level of cytokeratin is lower than about 0.04 (or the reference ratio of the reference activation level of PI3K protein to the reference expression level of cytokeratin. A responder to the biologic can have a ratio of activated PI3K protein to expression of cytokeratin protein that is less than about 0.04, e.g., about 0.039, about 0.030, about 0.020, about 0.010, about 0.009, or less.

C. Statistical Analysis

In some embodiments, a treatment selection model is established using a retrospective cohort with known outcomes of responders and non-responders to specific therapies. Statistical algorithms can be applied to the retrospective data on responders and non-responders. In some instances, logistic regression analysis is applied to determine statistically relevant reference levels, threshold levels or cut-off levels for one or more analytes described in the retrospective cohort. Also, treatment selection model can be combined or used with a logistic regression machine learning algorithm.

In certain instances, the statistical algorithm or statistical analysis is a learning statistical classifier system. In one aspect, the algorithm can be trained with known samples and thereafter validated with samples of known identity. As used herein, the term “learning statistical classifier system” includes a machine learning algorithmic technique capable of adapting to complex data sets (e.g., panel of markers of interest and/or list of IBS-related symptoms) and making decisions based upon such data sets. The learning statistical classifier system can be selected from the group consisting of a random forest (RF), classification and regression tree (C&RT), boosted tree, neural network (NN), support vector machine (SVM), general chi-squared automatic interaction detector model, interactive tree, multiadaptive regression spline, machine learning classifier, and combinations thereof. Preferably, the learning statistical classifier system is a tree-based statistical algorithm (e.g., RF, CART, etc.) and/or a NN (e.g., artificial NN, etc.). Additional examples of learning statistical classifier systems are described in U.S. Patent Application Publication Nos. 2008/0085524, 2011/0045476, and 2012/0171672.

In certain instances, the statistical algorithm is a single learning statistical classifier system. Preferably, the single learning statistical classifier system comprises a tree-based statistical algorithm such as a RF or CART. As a non-limiting example, a single learning statistical classifier system can be used to classify the sample as a therapy responder sample or therapy non-responder sample based upon a prediction or probability value and the expression and/or activation level of one or more analytes described herein, alone or in combination with the presence or absence of a disease endpoint (e.g., pathological complete response in breast and nodes). The use of a single learning statistical classifier system typically classifies the sample as a responder sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. As such, the classification of a sample as a therapy responder sample or non-responder sample is useful for aiding in treatment selection for a subject with breast cancer.

In certain other instances, the statistical algorithm is a combination of at least two learning statistical classifier systems. In some cases, the combination of learning statistical classifier systems comprises a RF and a NN, e.g., used in tandem or parallel. As a non-limiting example, a RF can first be used to generate a prediction or probability value based upon the diagnostic marker profile, alone or in combination with a symptom profile, and a NN can then be used to classify the sample as a responder sample or non-responder sample based upon the prediction or probability value and the same or different analyte profile or combination of profiles. In certain instances, the hybrid RF/NN learning statistical classifier system classifies the sample as a responder sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. In an exemplary embodiment, the statistical algorithm is a random forest classifier or a combination of a random forest classifier and a neural network classifier.

In some instances, the data obtained from using the learning statistical classifier system or systems can be processed using a processing algorithm. Such a processing algorithm can be selected, for example, from the group consisting of a multilayer perceptron, backpropagation network, and Levenberg-Marquardt algorithm. In other instances, a combination of such processing algorithms can be used, such as in a parallel or serial fashion.

In certain instances, the statistical algorithm or statistical analysis is a quartile analysis. In some cases, the quartile analysis converts the expression level or activation level of an analyte to a quartile score. As a non-limiting example, a determination of whether a human subject will be a responder or non-responder to a specific therapy can be made based upon a quartile sum score (QSS) that is obtained by summing the quartile score for a combination of detected analytes.

In quartile analysis, there are three numbers (values) that divide a range of data into four equal parts. The first quartile (also called the ‘lower quartile’) is the number below which lies the 25 percent of the bottom data. The second quartile (the ‘median’) divides the range in the middle and has 50 percent of the data below it. The third quartile (also called the ‘upper quartile’) has 75 percent of the data below it and the top 25 percent of the data above it. As a non-limiting example, quartile analysis can be applied to the expression level or activation level of an analyte described herein, such that an analyte level in the first quartile (<25%) is assigned a value of 1, an analyte level in the second quartile (25-50%) is assigned a value of 2, an analyte level in the third quartile (51%-<75%) is assigned a value of 3, and an analyte level in the fourth quartile (75%-100%) is assigned a value of 4.

The various statistical methods and models described herein can be trained and tested using a cohort of samples from therapy responders and therapy non-responders. One skilled in the art will know of additional techniques and drug selection criteria for obtaining a cohort of patient samples that can be used in training and testing the statistical methods and models described herein.

D. Administering Therapy Containing a Tyrosine Kinase Inhibitor or a Biologic

Provided herein is a method for administering a therapy comprising a tyrosine kinase inhibitor (TKI) to a subject who is likely to respond to the therapy. Non-limiting examples of a TKI include, without limitation, tyrosine kinase inhibitors such as neratinib, afatinib)(Gilotrif®, BIBW 2992, dacomitinib, poziotinib, lapatinib (GW-572016; Tykerb®), AZD8931 (sapaitinib), gefitinib (Iressa®), sunitinib (Sutent®), erlotinib (Tarceva®), canertinib (CI 1033), semaxinib (SU5416), vatalanib (PTK787/ZK222584), sorafenib (BAY 43-9006; Nexavar®), imatinib mesylate (Gleevec®), leflunomide (SU101), vandetanib (ZACTIMATM; ZD6474); and combinations thereof. A TKI can inhibit a receptor tyrosine kinase, a truncated receptor tyrosine kinase, a non-receptor tyrosine kinase, a heterodimer thereof, a homodimer thereof, or any combination thereof. In some embodiments, the TKI is a pan-HER inhibitor such as, but not limited to, neratinib, afatinib)(Gilotrif®, dacomitinib, poziotinib, AC480, or any combination thereof. In other embodiments, the TKI is a dual HER1/HER2 inhibitor such as, but not limited to, lapatinib, AZD8931, BIBW 2992, or any combination thereof

Provided herein is a method for administering a therapy comprising a biologic (an anti-cancer biologic) to a subject who is likely to respond to the therapy. Non-limiting examples of a biologic for treating breast cancer include, without limitation, monoclonal antibodies, affibodies, probodies, diabodies, dual antibodies, bispecific antibodies, fragments thereof, and combinations thereof. Affibodies include engineered proteins that can bind to a target protein(s) or peptide(s) with high binding affinity, thereby imitating the activity of monoclonal antibodies. A probody refers to an engineered proteolytically activated antibody (Desnoyers et al., Science Translational Medicine, 2013, 5(207): 207ra144; Polu and Louwman, Expt Opin Biol Ther, 2014, 14(5): 1049-53). Diabodies include, but are not limited to, small engineered bivalent and bispecific antibody fragments (Perisic, Structure, 1994, 2(12):1217-26). In some embodiments, the biologic for treating breast cancer is an anti-HER2 monoclonal antibody such as trastuzumab. In other embodiments, the biologic is an antibody that inhibits HER dimerization such as pertuzumab.

The therapy comprising a TKI or a biologic can be administered as neoadjuvant therapy. As such, the TKI or biologic can be administered to the subject with breast cancer prior to receiving surgery. In some cases, the TKI or biologic is administered in a treatment regimen that also includes paclitaxel, doxorubicin, cyclophosphamide, or combinations thereof

### IV. EXAMPLE

The following example is offered to illustrate, but not to limit, the claimed invention.

### Example 1

### HER2 and HER3 Signaling Pathway Biomarkers Can Predict Responses of Breast Cancer Tumors to Anti-Cancer Drugs Such as Tyrosine Kinases Inhibitors and Anti-Cancer Biologics

This example illustrates that the expression level and/or activation level of one or more signal transduction molecules of the HER2 and HER3 signaling pathways can be used to determine if a patient is likely to respond to a tyrosine kinase inhibitor or a biologic for the treatment of breast cancer. Also described are study results that support such a method.

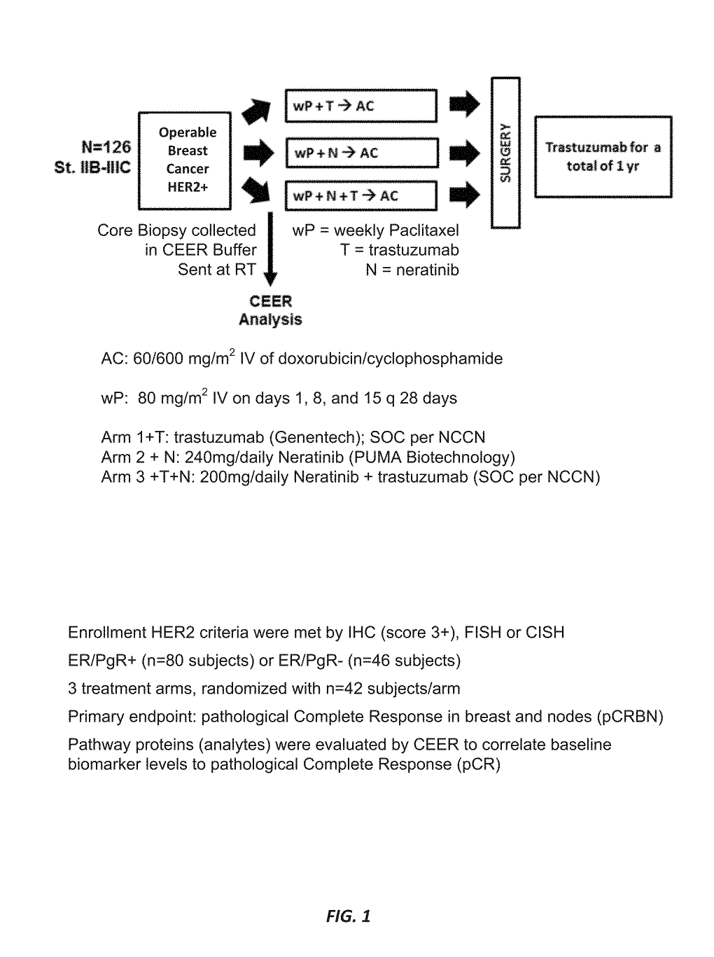

In a Phase II, multi-center randomized study, women with HER2-positive, locally advanced breast cancer were treated with neratinib in combination with weekly paclitaxel with or without trastuzumab, followed by doxorubicin and cyclophosphamide (AC) as neoadjuvant therapy. Patients in the control arm received neoadjuvant trastuzumab in combination with weekly paclitaxel followed by AC. FIG. 1 provides a schematic diagram of the clinical study.

Patients in Arm 1 received 4 cycles of paclitaxel 80 mg/m² administered on Days 1, 8, and 15 of a 28-day cycle. Trastuzumab was begun concurrently with paclitaxel and was given weekly for a total of 16 doses (4 mg/kg loading dose, then 2 mg/kg weekly). Following paclitaxel/trastuzumab, standard AC (60/600 mg/m² IV of doxorubicin/ cyclophosphamide) was administered every 21 days for 4 cycles.

Patients in Arm 2 received 4 cycles of paclitaxel 80 mg/m² administered on Days 1, 8, and 15 of a 28-day cycle. Neratinib 240 mg was taken orally once daily beginning on Day 1 of paclitaxel and continuing through Day 28 of the final cycle of paclitaxel. Standard AC was administered every 21 days for 4 cycles, and then it was administered following paclitaxel/neratinib therapy.

Patients in Arm 3 received 4 cycles of paclitaxel 80 mg/ m² administered Days 1, 8, and 15 of a 28 day cycle with trastuzumab, beginning concurrently with paclitaxel, given weekly for a total of 16 doses (4 mg/kg loading dose, then 2 mg/kg weekly). Neratinib 200 mg was taken orally once daily beginning on Day 1 of paclitaxel and continuing through Day 28 of the final cycle of paclitaxel. Standard AC was administered every 21 days for 4 cycles following paclitaxel/trastuzumab/neratinib therapy.

In all arms, clinical response was assessed by palpation between the chemotherapy regimens and prior to surgery. Clinical data and status of pathological complete response (pCR) was collected for these patients.

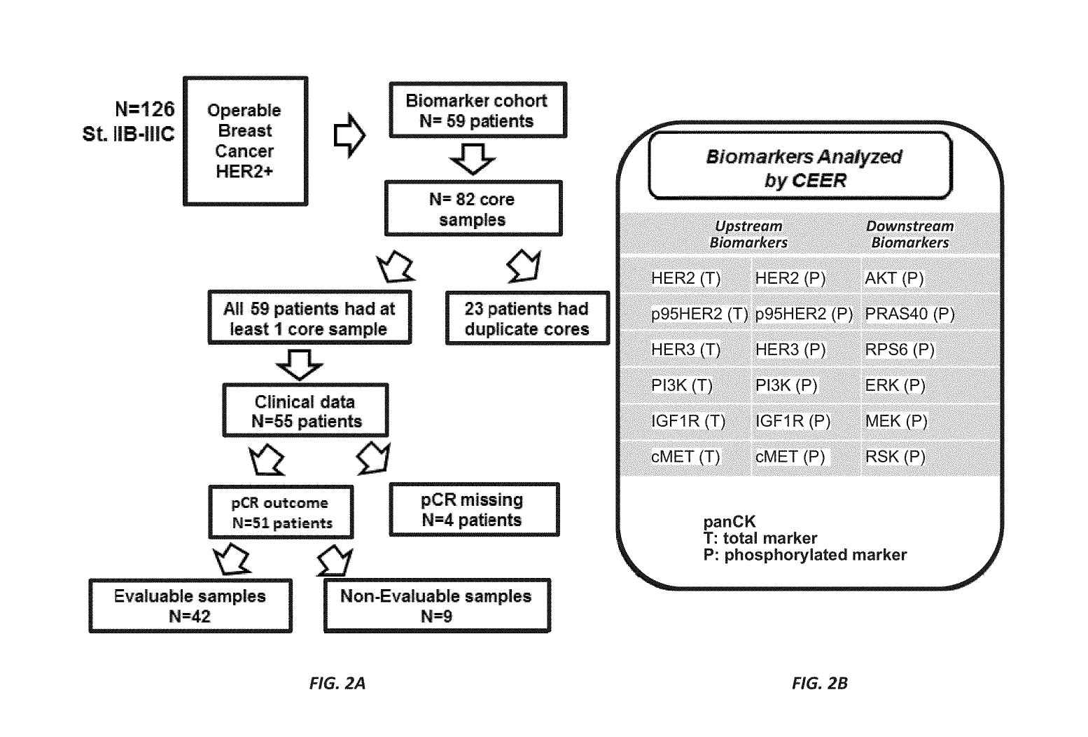

Three fresh tumor samples from subjects participating in the CEER study were obtained before treatment randomization and before the start of the study therapy. A blood sample was also collected before the start of the study therapy. Samples from a total of 42 patients were analyzed (FIG. 2A). These patients had operable breast cancer (stage IIB-IIIC) and were HER2 positive, according to an immunohistochemistry score of 3+ and/or a FISH score of positive. Clinical data and status of pCR outcome was collected for these patients.

In the present study, the expression level and/or activation level of specific biomarkers in baseline samples obtained from the study patients were analyzed by CEER™. For example, the total expression level of HER2 protein, the total expression level of p95HER2 protein, the total expression level of p95HER2 protein, the total expression level of HER3 protein, the total expression level of PI3K protein, the total expression level of IGF1R protein, the total expression level of cMET protein, the activation level of HER2 protein, the activation level of p95HER2 protein, the activation level of p95HER2 protein, the activation level of HER3 protein, the activation level of PI3K protein, the activation level of IGF1R protein, the activation level of cMET protein, the activation level of AKT protein, the activation level of PRAS40 protein, the activation level of RPS6 protein, the activation level of ERK1 protein, the activation level of MEK1 protein, the activation level of RSK protein, and the total expression level of cytokeratin (CK or panCK) were quantitated in the samples (FIG. 2B). The data show a wide range of expression level of HER2 protein in HER2 positive tumor samples.

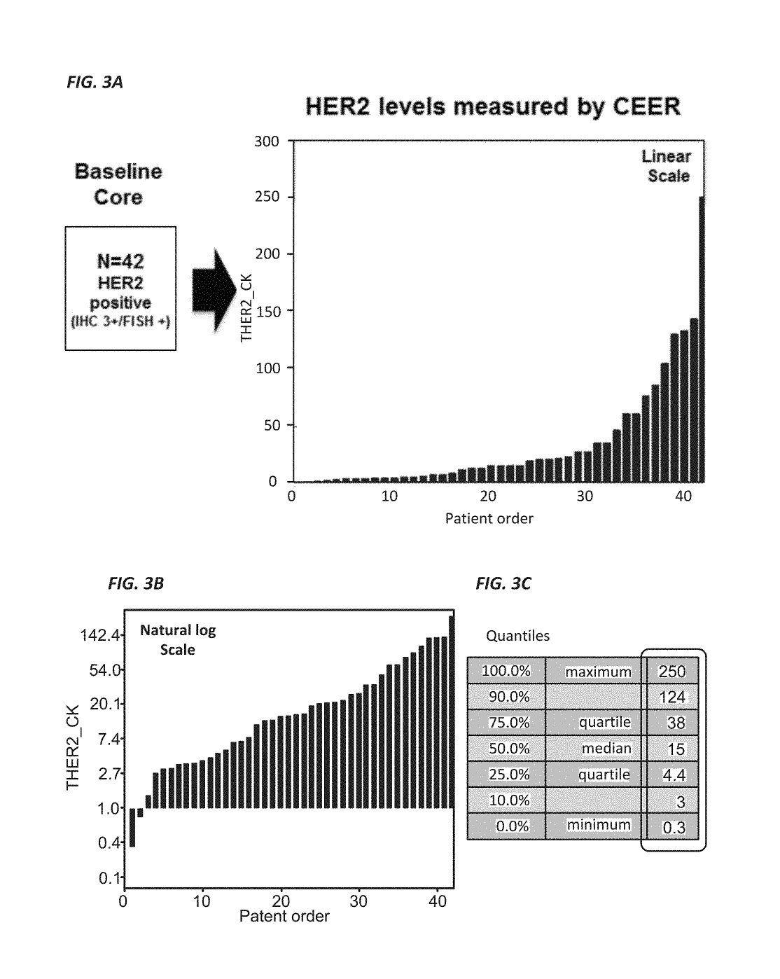

The distribution of HER2 expression levels (a ratio of total HER2 level to CK level) in the samples is provided in FIG. 3A. The same data is presented in natural log scale in FIG. 3B. Statistical analysis of the distribution according to quantiles and median is shown in FIG. 3C. The maximum level of HER2 expression at baseline in the samples was 250; the median level was 15; and the minimum level was 0.3. For the top quartile spanning from 75%-100% quantiles, the expression level of HER2 ranged from 38-250. For the lowest quartile spanning from 0%-25% quantiles, the level was from 0.3-4.4.

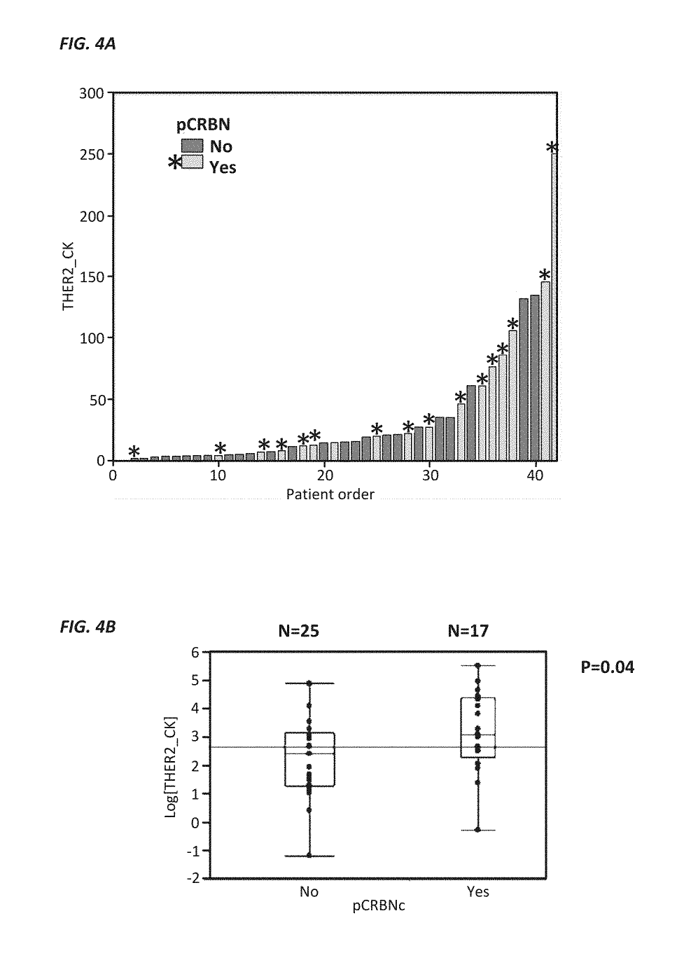

FIG. 4A correlates the expression level of HER2 protein with the patient's clinical outcome (e.g., the presence of pathological complete response with treatment such as HER2-targeted treatment). About 60% of HER2 positive patients (24 of 42 patients) did not achieve pathological complete response in breast and nodes (pCRBN) when administered HER2-targeted treatment (FIG. 4B). Patients considered non-responders (those who did not achieve pCRBN) had low levels of HER2 expression relative to all patients analyzed.

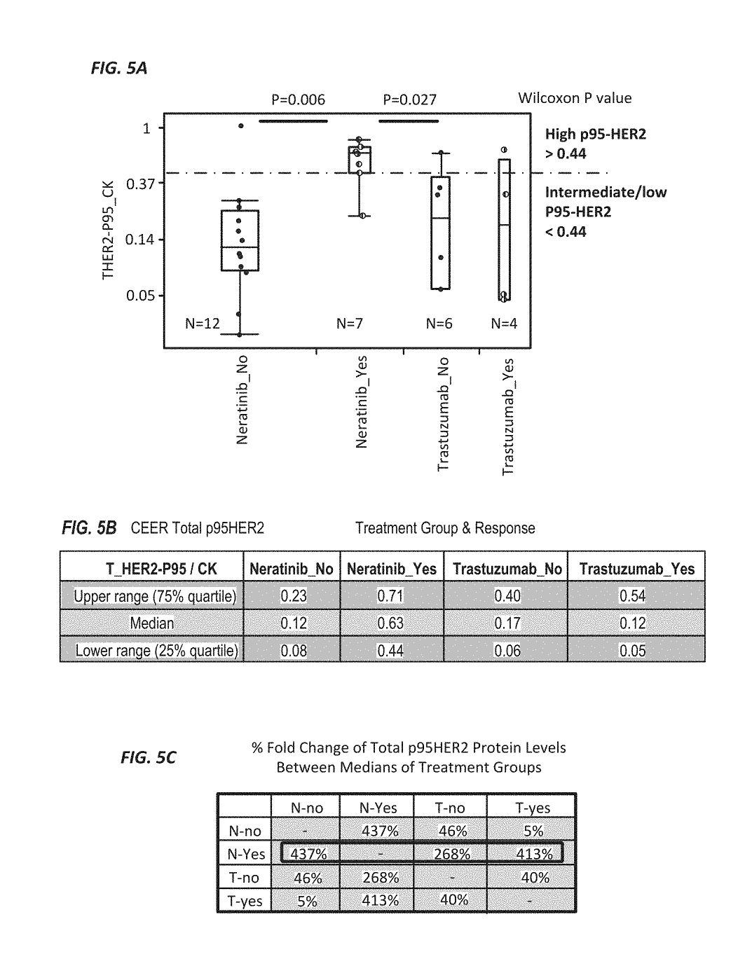

The data shows that patients who responded to a tyrosine kinase inhibitor that targets multiple HER signaling pathways (i.e., neratinib) had tumors with elevated expression levels and/or activation levels of signal transduction molecules of HER2 signaling pathway. Responders to neratinib had tumors with higher levels of truncated HER2 compared to non-responders of neratinib (FIGS. 5A, 5B and 5C). As such, patients with a high level of p95HER2 , or a ratio of p95HER2 protein expression to CK protein expression greater than 0.44 are likely to respond to neratinib or other tyrosine kinase inhibitors. Patients with a ratio less than 0.44 or having an intermediate/low level of p95HER2 protein expression are predicted to be non-responders of neratinib. FIG. 5C shows the percent (%) fold change of truncated HER2 protein levels between medians in the treatment groups (responders and non-responders to neratinib, and responders and non-responders to trastuzumab). Neratinib responders have a 3-fold to 5-fold higher expression of p95HER2 than those in the other treatment groups.

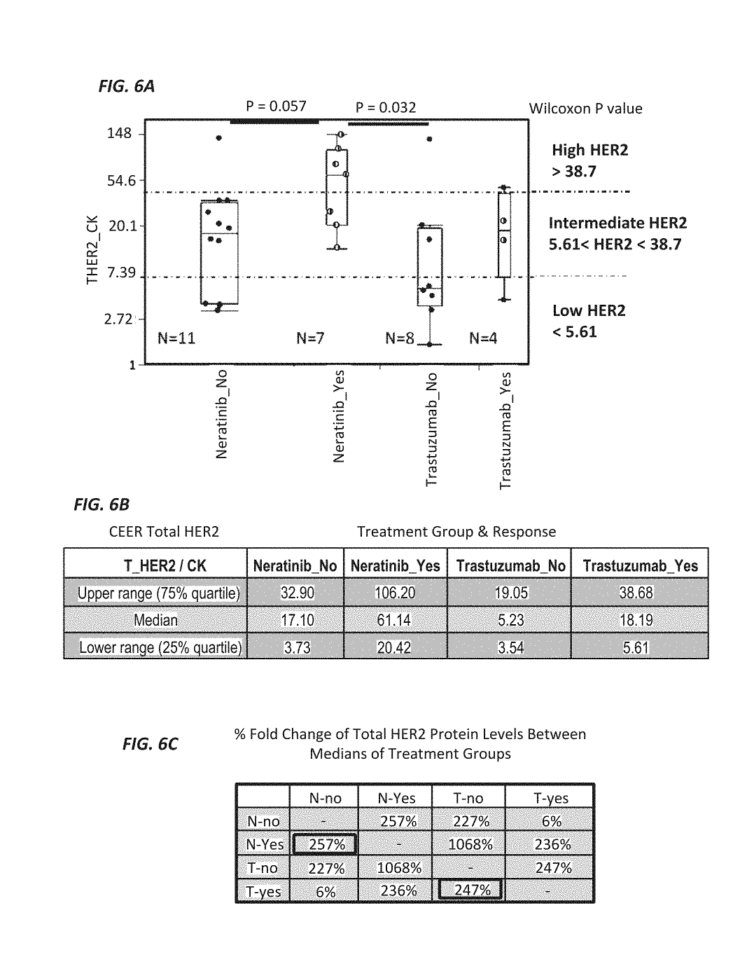

Responders to neratinib also had tumors with high levels of full-length HER2 protein compared to non-responders of neratinib (FIGS. 6A, 6B and 6C). These responders had a ratio of full-length HER2 protein to CK protein expression of greater than 38.7. FIG. 6C shows the % fold change of full-length HER2 protein levels between medians in the treatment groups (responders and non-responders to neratinib, and responders and non-responders to trastuzumab). Neratinib responders have a 2.5-fold higher expression of full-length HER2 protein than those in the other groups. These TKI responders had tumors with the highest level HER2 protein (or an equivalent to a ratio of greater than 38.7). Patients with tumors with an intermediate level of HER2 protein or a ratio of full-length HER2 protein to CK protein expression between 5.61 and 38.7 responded to trastuzumab, and did not respond to neratinib. Patients with tumors with a low level of HER2 protein or a ratio of full-length HER2 protein to CK protein expression of less than 5.61 did not respond to trastuzumab or neratinib.

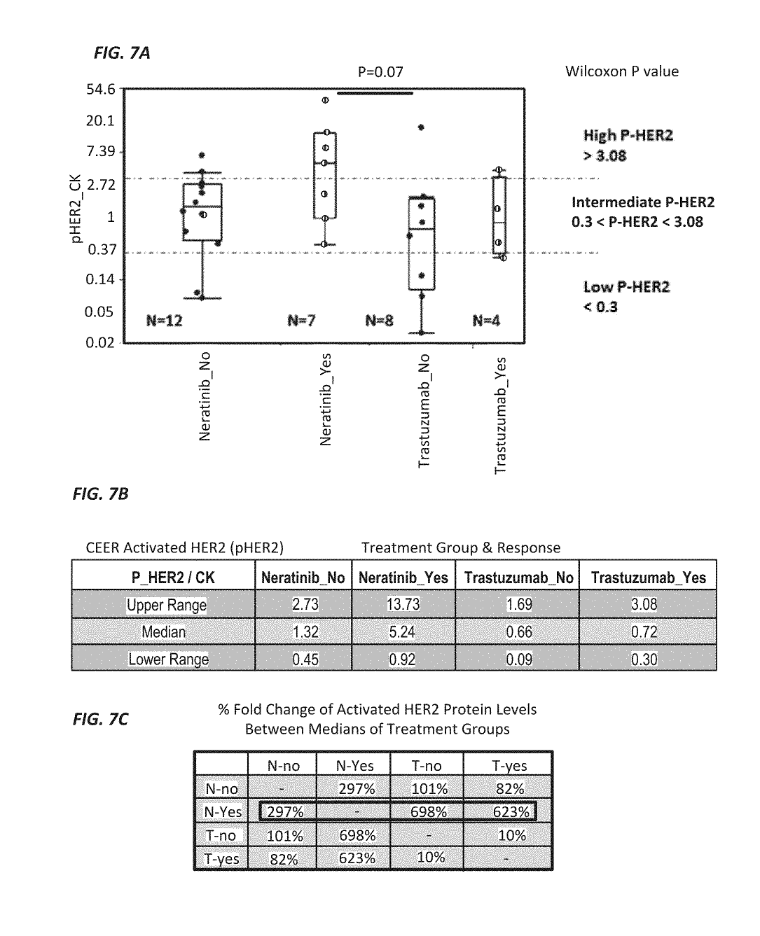

Patients with a high activation level of HER2 protein or a ratio of activated HER2 level to expression level of CK that is greater than 3.08 responded to neratinib (FIGS. 7A, 7B and 7C). Patients with tumors that express a high level of HER2 protein at baseline (before receiving a drug therapy) are predicted to respond to a TKI such as neratinib. Within each treatment group (neratinib group or trastuzumab group), the median level of HER2 expression at baseline was higher for responders than non-responders.

The study also shows that some patients exhibited tumors that responded to the biologic trastuzumab. These tumors were characterized as having an intermediate level of HER2 expression compared to those who were responders to neratinib and had high levels of HER2 expression (FIG. 6A).

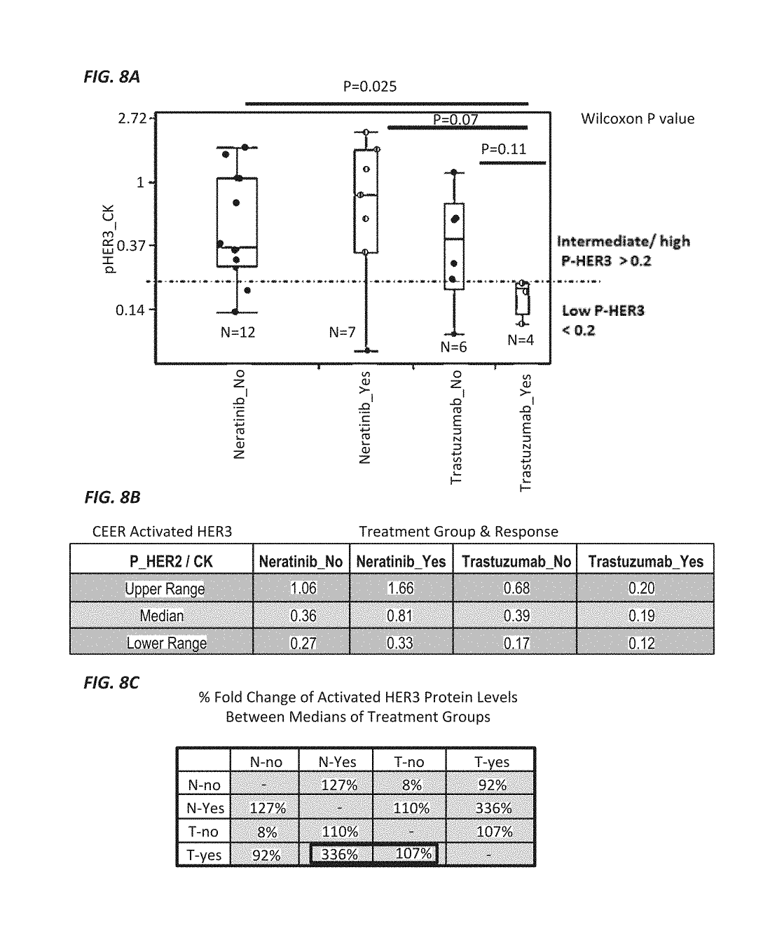

Patient tumors with a low level of activated HER3 protein or a ratio of activated HER3 level to expression level of CK that is less than 0.2 were responsive to trastuzumab treatment. Patients with a high level of activated HER3 protein (a ratio of greater than 0.2) did not respond to trastuzumab (FIGS. 8A, 8B and 8C). Responders to TKI also had a higher level of activated HER3 protein compared to non-responders.

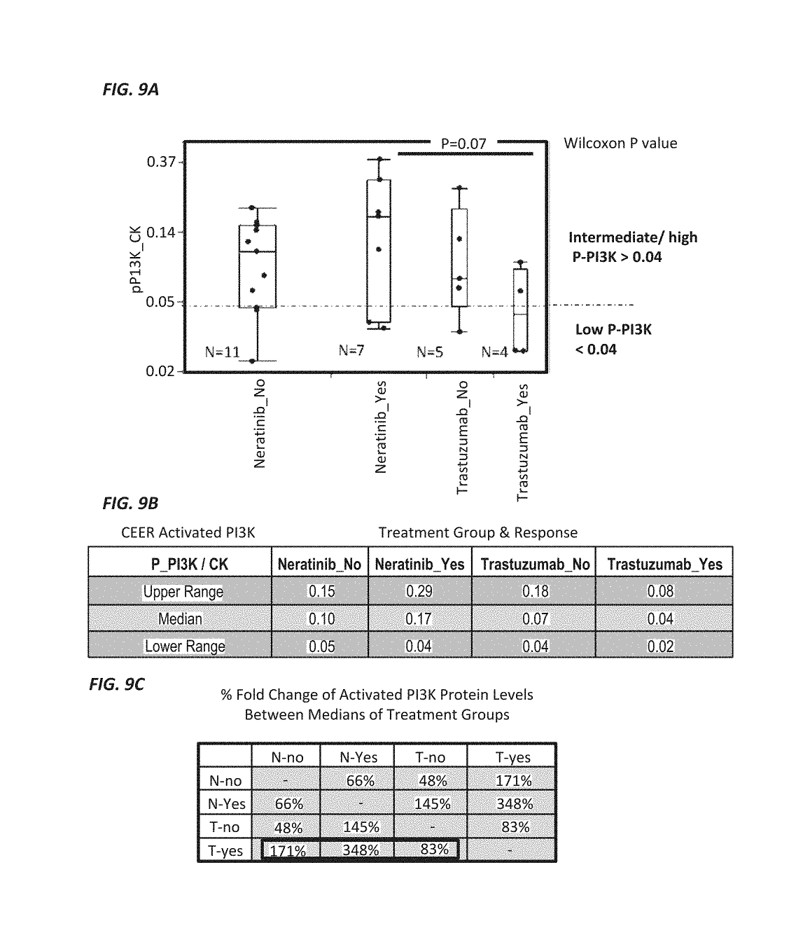

Responders to trastuzumab treatment had low levels of activated PI3K protein or a ratio of activated PI3K levels to expression level of CK of less than 0.04. This supports published data showing that high activated PI3K levels lead to resistance to trastuzumab. Tumors with high levels of activated PI3K were responsive to neratinib (FIGS. 9A, 9B and 9C).

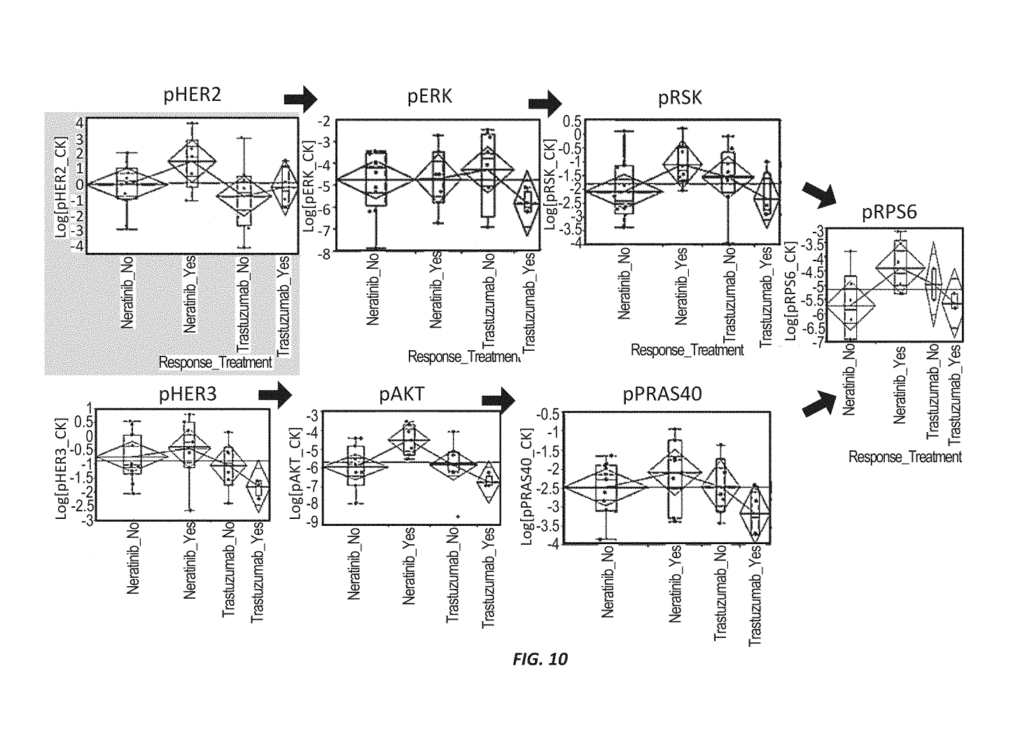

Analysis of other HER signaling transduction molecules including activated AKT, activated PRAS40, activated ERK, and activated RSK showed that responders to trastuzumab expressed low levels of these molecule compared to trastuzumab non-responders and neratinib non-responders (FIG. 10).

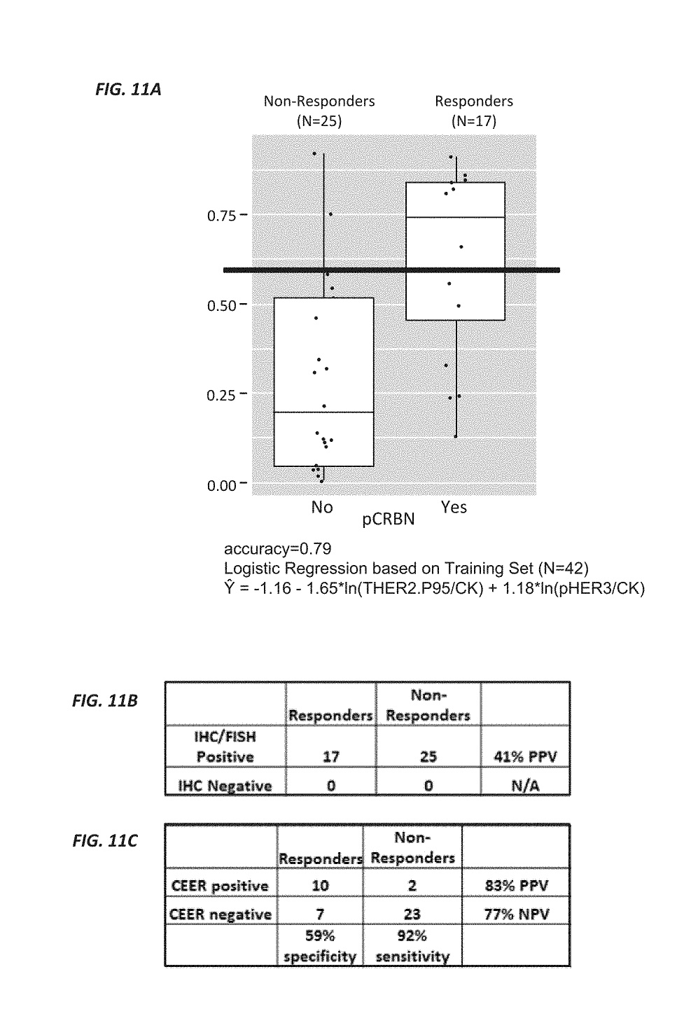

The expression levels and/or activation levels of multiple signaling transduction molecules can be combined into a statistical model to determine which patients are responders or non-responders to specific therapies, e.g., neratinib or trastuzumab. This statistical model can be used predict the likelihood of treatment response in patients who have not received the treatment. FIGS. 11A, 11B and 11C show that expression levels of truncated HER2 protein were combined with activation levels of HER3 protein and transformed using an algorithm to separate responders and non-responders to trastuzumab. FIG. 11A shows that non-responders had a profile or score below a selected cut-off value (0.60; CEER negative) and responders were CEER positive or had a profile or score greater than the cut-off In this exemplary statistical model of the data, the method for determining treatment response had 59% specificity and 92% sensitivity.

### V. EXEMPLARY EMBODIMENTS

Exemplary embodiments provided in accordance with the presently disclosed subject matter include, but are not limited to, the claims and the following embodiments:
1. A method for determining whether a human subject with breast cancer will respond to therapy with a tyrosine kinase inhibitor or a biologic, the method comprising:
   (a) lysing a breast cancer cell obtained from a sample from the human subject to produce a cellular extract;
   (b) determining an expression level of truncated HER2 protein, an expression level of full-length HER2 protein, an activation level of full-length HER2 protein, an activation level of HER3 protein, and/or an activation level of PI3K protein in the cellular extract;
   (c) comparing the expression level of truncated HER2 protein, the expression level of full-length HER2 protein, the activation level of full-length HER2 protein, the activation level of HER3 protein, and/or the activation level of PI3K protein in the cellular extract to a reference expression level of truncated HER2 protein, a reference expression level of full-length HER2 protein, a reference activation level of full-length HER2 protein, a reference activation level of HER3 protein, and/or a reference activation level of PI3K protein, and
   (d) determining whether a human subject with breast cancer will respond to therapy with a tyrosine kinase inhibitor or a biologic based upon a difference between the expression level of truncated HER2 protein, the expression level of full-length HER2 protein, the activation level of full-length HER2 protein, the activation level of HER3 protein, and/or the activation level of PI3K protein in the cellular extract compared to the reference expression level of truncated HER2 protein, the reference expression level of full-length HER2 protein, the reference activation level of full-length HER2 protein, the reference activation level of HER3 protein, and/or the reference activation level of PI3K protein.
2. The method of embodiment 1, wherein the breast cancer is HER2-positive, locally advanced breast cancer.
3. The method of embodiment 1 or 2, wherein the tyrosine kinase inhibitor is a pan-HER inhibitor or a dual HER1/HER2 inhibitor.
4. The method of embodiment 3, wherein the pan-HER inhibitor is selected from the group consisting of neratinib, afatinib, dacomitinib, poziotinib, and combinations thereof.
5. The method of embodiment 3, wherein the dual HER1/HER2 inhibitor is selected from the group consisting of lapatinib, AZD8931, BIBW 2992, and combinations thereof.
6. The method of any one of embodiments 1 to 5, wherein the biologic is selected from the group consisting of a monoclonal antibody, an affibody, a probody, a diabody, a dual antibody, fragments thereof, and combinations thereof.
7. The method of embodiment 6, wherein the monoclonal antibody is an anti-HER2 antibody or an antibody that inhibits HER dimerization.
8. The method of embodiment 7, wherein the anti-HER2 antibody is trastuzumab.
9. The method of embodiment 7, wherein the antibody that inhibits HER dimerization is pertuzumab.
10. The method of any one of embodiments 1 to 9, wherein the therapy is used as neoadjuvant therapy.
11. The method of embodiment 10, wherein the neoadjuvant therapy further comprises paclitaxel, doxorubicin, cyclophosphamide, or combinations thereof.
12. The method of any one of embodiments 1 to 11, wherein the human subject will likely respond to therapy with a tyrosine kinase inhibitor when the expression level of truncated HER2 protein in the cellular extract is higher than the reference expression level of truncated HER2 protein.
13. The method of embodiment 12, wherein the reference expression level of truncated HER2 protein is a median expression level of truncated HER2 protein in human subjects who did not respond to the tyrosine kinase inhibitor, in human subjects who did not respond to the biologic, and/or in human subjects who responded to the biologic.
14. The method of embodiment 13, wherein the human subject will likely respond to therapy with the tyrosine kinase inhibitor when the expression level of truncated HER2 protein in the cellular extract is about 3-fold to about 5-fold higher than the median expression level of truncated HER2 protein.
15. The method of embodiment 12, wherein the expression level of truncated HER2 protein in the cellular extract is a ratio of the expression level of truncated HER2 protein in the cellular extract to an expression level of a control protein.
16. The method of embodiment 15, wherein the control protein is cytokeratin (CK).
17. The method of embodiment 16, wherein the human subject will likely respond to therapy with the tyrosine kinase inhibitor when the expression level of truncated HER2 protein in the cellular extract is higher than a reference expression level of truncated HER2 protein corresponding to a ratio of about 0.44 relative to the expression level of CK.
18. The method of any one of embodiments 1 to 17, wherein the human subject will likely respond to therapy with either a tyrosine kinase inhibitor or a biologic when the expression level of full-length HER2 protein in the cellular extract is higher than the reference expression level of full-length HER2 protein.
19. The method of embodiment 18, wherein the reference expression level of full-length HER2 protein is a median expression level of full-length HER2 protein in human subjects who did not respond to the tyrosine kinase inhibitor.
20. The method of embodiment 19, wherein the human subject will likely respond to therapy with the tyrosine kinase inhibitor when the expression level of full-length HER2 protein in the cellular extract is about 2.5-fold higher than the median expression level of full-length HER2 protein.
21. The method of embodiment 18, wherein the reference expression level of full-length HER2 protein is a median expression level of full-length HER2 protein in human subjects who did not respond to the biologic.
22. The method of embodiment 21, wherein the human subject will likely respond to therapy with the biologic when the expression level of full-length HER2 protein in the cellular extract is about 2.5-fold higher than the median expression level of full-length HER2 protein.
23. The method of embodiment 18, wherein the expression level of full-length HER2 protein in the cellular extract is a ratio of the expression level of full-length HER2 protein in the cellular extract to an expression level of a control protein.
24. The method of embodiment 23, wherein the control protein is cytokeratin (CK).
25. The method of embodiment 24, wherein the human subject will likely respond to therapy with the tyrosine kinase inhibitor when the expression level of full-length HER2 protein in the cellular extract is higher than a reference expression level of full-length HER2 protein corresponding to a ratio of about 38.7 relative to the expression level of CK.
26. The method of embodiment 24, wherein the human subject will likely respond to therapy with the biologic when the expression level of full-length HER2 protein in the cellular extract is between a reference expression level of full-length HER2 protein corresponding to a ratio of from about 5.6 to about 38.7 relative to the expression level of CK.
27. The method of embodiment 24, wherein the human subject will likely not respond to therapy with either the tyrosine kinase inhibitor or the biologic when the expression level of full-length HER2 protein in the cellular extract is lower than a reference expression level of full-length HER2 protein corresponding to a ratio of about 5.6 relative to the expression level of CK.
28. The method of any one of embodiments 1 to 27, wherein the human subject will likely respond to therapy with a tyrosine kinase inhibitor when the activation level of full-length HER2 protein in the cellular extract is higher than the reference activation level of full-length HER2 protein.
29. The method of embodiment 28, wherein the reference activation level of full-length HER2 protein is a median activation level of full-length HER2 protein in human subjects who did not respond to the tyrosine kinase inhibitor, in human subjects who did not respond to the biologic, and/or in human subjects who responded to the biologic.
30. The method of embodiment 29, wherein the human subject will likely respond to therapy with the tyrosine kinase inhibitor when the activation level of full-length HER2 protein in the cellular extract is about 3-fold to about 7-fold higher than the median activation level of full-length HER2 protein.
31. The method of embodiment 28, wherein the activation level of full-length HER2 protein in the cellular extract is a ratio of the activation level of full-length HER2 protein in the cellular extract to an expression level of a control protein.
32. The method of embodiment 31, wherein the control protein is cytokeratin (CK).
33. The method of embodiment 32, wherein the human subject will likely respond to therapy with the tyrosine kinase inhibitor when the activation level of full-length HER2 protein in the cellular extract is higher than a reference activation level of full-length HER2 protein corresponding to a ratio of about 3.1 relative to the expression level of CK.
34. The method of embodiment 33, wherein the human subject will likely not respond to therapy with either the tyrosine kinase inhibitor or the biologic when the activation level of full-length HER2 protein in the cellular extract is lower than a reference activation level of full-length HER2 protein corresponding to a ratio of about 0.3 relative to the expression level of CK.
35. The method of any one of embodiments 1 to 34, wherein the human subject will likely respond to therapy with a tyrosine kinase inhibitor when the activation level of HER3 protein in the cellular extract is higher than the reference activation level of HER3 protein.
36. The method of embodiment 35, wherein the reference activation level of HER3 protein is a median activation level of HER3 protein in human subjects who did not respond to the tyrosine kinase inhibitor, in human subjects who did not respond to the biologic, and/or in human subjects who responded to the biologic.
37. The method of embodiment 36, wherein the human subject will likely respond to therapy with a tyrosine kinase inhibitor when the activation level of HER3 protein in the cellular extract is about 1-fold to about 3.5-fold higher than the median activation level of HER3 protein.
38. The method of any one of embodiments 1 to 34, wherein the human subject will likely respond to therapy with a biologic when the activation level of HER3 protein in the cellular extract is lower than the reference activation level of HER3 protein.
39. The method of embodiment 38, wherein the reference activation level of HER3 protein is a median activation level of HER3 protein in human subjects who did not respond to the biologic, in human subjects who did not respond to the tyrosine kinase inhibitor, and/or in human subjects who responded to the tyrosine kinase inhibitor.
40. The method of embodiment 39, wherein the human subject will likely respond to therapy with a biologic when the activation level of HER3 protein in the cellular extract is about 1-fold to about 3.5-fold lower than the median activation level of HER3 protein.
41. The method of embodiment 35 or 38, wherein the activation level of HER3 protein in the cellular extract is a ratio of the activation level of HER3 protein in the cellular extract to an expression level of a control protein.
42. The method of embodiment 41, wherein the control protein is cytokeratin (CK).
43. The method of embodiment 42, wherein the human subject will likely respond to therapy with the tyrosine kinase inhibitor when the activation level of HER3 protein in the cellular extract is higher than a reference activation level of HER3 protein corresponding to a ratio of about 0.2 relative to the expression level of CK.
44. The method of embodiment 42, wherein the human subject will likely respond to therapy with the biologic when the activation level of HER3 protein in the cellular extract is lower than a reference activation level of HER3 protein corresponding to a ratio of about 0.2 relative to the expression level of CK.
45. The method of any one of embodiments 1 to 44, wherein the human subject will likely respond to therapy with a tyrosine kinase inhibitor when the activation level of PI3K protein in the cellular extract is higher than the reference activation level of PI3K protein.
46. The method of embodiment 45, wherein the reference activation level of PI3K protein is a median activation level of PI3K protein in human subjects who did not respond to the tyrosine kinase inhibitor, in human subjects who did not respond to the biologic, and/or in human subjects who responded to the biologic.
47. The method of embodiment 46, wherein the human subject will likely respond to therapy with a tyrosine kinase inhibitor when the activation level of PI3K protein in the cellular extract is about 0.5-fold to about 3.5-fold higher than the median activation level of PI3K protein.
48. The method of any one of embodiments 1 to 44, wherein the human subject will likely respond to therapy with a biologic when the activation level of PI3K protein in the cellular extract is lower than the reference activation level of PI3K protein.
49. The method of embodiment 48, wherein the reference activation level of PI3K protein is a median activation level of PI3K protein in human subjects who did not respond to the biologic, in human subjects who did not respond to the tyrosine kinase inhibitor, and/or in human subjects who responded to the tyrosine kinase inhibitor.
50. The method of embodiment 49, wherein the human subject will likely respond to therapy with a biologic when the activation level of PI3K protein in the cellular extract is about 0.5-fold to about 3.5-fold lower than the median activation level of PI3K protein.
51. The method of embodiment 45 or 48, wherein the activation level of PI3K protein in the cellular extract is a ratio of the activation level of PI3K protein in the cellular extract to an expression level of a control protein.
52. The method of embodiment 51, wherein the control protein is cytokeratin (CK).
53. The method of embodiment 52, wherein the human subject will likely respond to therapy with the tyrosine kinase inhibitor when the activation level of PI3K protein in the cellular extract is higher than a reference activation level of PI3K protein corresponding to a ratio of about 0.04 relative to the expression level of CK.
54. The method of embodiment 52, wherein the human subject will likely respond to therapy with the biologic when the activation level of PI3K protein in the cellular extract is lower than a reference activation level of PI3K protein corresponding to a ratio of about 0.04 relative to the expression level of CK.
55. The method of embodiment 1, wherein the expression level of truncated HER2 protein, the expression level of full-length HER2 protein, and the activation level of full-length HER2 protein is determined.
56. The method of embodiment 1, wherein the expression level of full-length HER2 protein and the activation level of HER3 protein is determined.
57. The method of any one of embodiments 1 to 56, wherein the method further comprises determining an expression level and/or an activation level of one or more additional signal transduction molecules in the cellular extract.
58. The method of embodiment 57, wherein the one or more additional signal transduction molecules is selected from the group consisting of AKT, PRAS40, ERK1 (MAPK3), ERK2 (MAPK1), RSK, and combinations thereof.
59. The method of any one of embodiments 1 to 58, wherein the sample is a breast tumor tissue, whole blood, serum, or plasma sample.
60. The method of embodiment 59, wherein the breast tumor tissue sample is a needle biopsy sample.
61. The method of any one of embodiments 1 to 60, wherein the expression level of truncated HER2 protein, the expression level of full-length HER2 protein, the activation level of full-length HER2 protein, the activation level of HER3 protein, and/or the activation level of PI3K protein is determined with Collaborative Enzyme Enhanced Reactive ImmunoAssay (CEER).
62. The method of any one of embodiments 1 to 61, wherein the method further comprises administering the tyrosine kinase inhibitor or the biologic to the human subject.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for determining whether a human subject with breast cancer will respond to therapy with a tyrosine kinase inhibitor or a biologic, the method comprising:
(a) lysing a breast cancer cell obtained from a sample from the human subject to produce a cellular extract;
(b) determining an expression level of truncated HER2 protein, an expression level of full-length HER2 protein, an activation level of full-length HER2 protein, an activation level of HER3 protein, and/or an activation level of PI3K protein in the cellular extract;
(c) comparing the expression level of truncated HER2 protein, the expression level of full-length HER2 protein, the activation level of full-length HER2 protein, the activation level of HER3 protein, and/or the activation level of PI3K protein in the cellular extract to a reference expression level of truncated HER2 protein, a reference expression level of full-length HER2 protein, a reference activation level of full-length HER2 protein, a reference activation level of HER3 protein, and/or a reference activation level of PI3K protein, and
(d) determining whether a human subject with breast cancer will respond to therapy with a tyrosine kinase inhibitor or a biologic based upon a difference between the expression level of truncated HER2 protein, the expression level of full-length HER2 protein, the activation level of full-length HER2 protein, the activation level of HER3 protein, and/or the activation level of PI3K protein in the cellular extract compared to the reference expression level of truncated HER2 protein, the reference expression level of full-length HER2 protein, the reference activation level of full-length HER2 protein, the reference activation level of HER3 protein, and/or the reference activation level of PI3K protein.

2. The method of claim 1, wherein the breast cancer is HER2-positive, locally advanced breast cancer.

3. The method of claim 1, wherein the tyrosine kinase inhibitor is a pan-HER inhibitor or a dual HER1/HER2 inhibitor.

4. The method of claim 3, wherein the pan-HER inhibitor is selected from the group consisting of neratinib, afatinib, dacomitinib, poziotinib, and combinations thereof.

5. The method of claim 3, wherein the dual HER1/HER2 inhibitor is selected from the group consisting of lapatinib, AZD8931, BIBW 2992, and combinations thereof.

6. The method of claim 1, wherein the biologic is selected from the group consisting of a monoclonal antibody, an affibody, a probody, a diabody, a dual antibody, fragments thereof, and combinations thereof.

7. The method of claim 6, wherein the monoclonal antibody is an anti-HER2 antibody or an antibody that inhibits HER dimerization.

8. The method of claim 7, wherein the anti-HER2 antibody is trastuzumab.

9. The method of claim 7, wherein the antibody that inhibits HER dimerization is pertuzumab.

10. The method of claim 1, wherein the therapy is used as neoadjuvant therapy.

11. The method of claim 10, wherein the neoadjuvant therapy further comprises paclitaxel, doxorubicin, cyclophosphamide, or combinations thereof.

12. The method of claim 1, wherein the human subject will likely respond to therapy with a tyrosine kinase inhibitor when the expression level of truncated HER2 protein in the cellular extract is higher than the reference expression level of truncated HER2 protein.

13. The method of claim 12, wherein the reference expression level of truncated HER2 protein is a median expression level of truncated HER2 protein in human subjects who did not respond to the tyrosine kinase inhibitor, in human subjects who did not respond to the biologic, and/or in human subjects who responded to the biologic.

14. The method of claim 13, wherein the human subject will likely respond to therapy with the tyrosine kinase inhibitor when the expression level of truncated HER2 protein in the cellular extract is about 3-fold to about 5-fold higher than the median expression level of truncated HER2 protein.

15. The method of claim 12, wherein the expression level of truncated HER2 protein in the cellular extract is a ratio of the expression level of truncated HER2 protein in the cellular extract to an expression level of a control protein.

16. The method of claim 15, wherein the control protein is cytokeratin (CK).

17. The method of claim 16, wherein the human subject will likely respond to therapy with the tyrosine kinase inhibitor when the expression level of truncated HER2 protein in the cellular extract is higher than a reference expression level of truncated HER2 protein corresponding to a ratio of about 0.44 relative to the expression level of CK.

18. The method of claim 1, wherein the human subject will likely respond to therapy with either a tyrosine kinase inhibitor or a biologic when the expression level of full-length HER2 protein in the cellular extract is higher than the reference expression level of full-length HER2 protein.

19. The method of claim 18, wherein the reference expression level of full-length HER2 protein is a median expression level of full-length HER2 protein in human subjects who did not respond to the tyrosine kinase inhibitor.

20. The method of claim 19, wherein the human subject will likely respond to therapy with the tyrosine kinase inhibitor when the expression level of full-length HER2 protein in the cellular extract is about 2.5-fold higher than the median expression level of full-length HER2 protein.

21. The method of claim 18, wherein the reference expression level of full-length HER2 protein is a median expression level of full-length HER2 protein in human subjects who did not respond to the biologic.

22. The method of claim 21, wherein the human subject will likely respond to therapy with the biologic when the expression level of full-length HER2 protein in the cellular extract is about 2.5-fold higher than the median expression level of full-length HER2 protein.

23. The method of claim 18, wherein the expression level of full-length HER2 protein in the cellular extract is a ratio of the expression level of full-length HER2 protein in the cellular extract to an expression level of a control protein.

24. The method of claim 23, wherein the control protein is cytokeratin (CK).

25. The method of claim 24, wherein the human subject will likely respond to therapy with the tyrosine kinase inhibitor when the expression level of full-length HER2 protein in the cellular extract is higher than a reference expression level of full-length HER2 protein corresponding to a ratio of about 38.7 relative to the expression level of CK.

26. The method of claim 24, wherein the human subject will likely respond to therapy with the biologic when the expression level of full-length HER2 protein in the cellular extract is between a reference expression level of full-length HER2 protein corresponding to a ratio of from about 5.6 to about 38.7 relative to the expression level of CK.

27. The method of claim 24, wherein the human subject will likely not respond to therapy with either the tyrosine kinase inhibitor or the biologic when the expression level of full-length HER2 protein in the cellular extract is lower than a reference expression level of full-length HER2 protein corresponding to a ratio of about 5.6 relative to the expression level of CK.

28. The method of claim 1, wherein the human subject will likely respond to therapy with a tyrosine kinase inhibitor when the activation level of full-length HER2 protein in the cellular extract is higher than the reference activation level of full-length HER2 protein.

29. The method of claim 28, wherein the reference activation level of full-length HER2 protein is a median activation level of full-length HER2 protein in human subjects who did not respond to the tyrosine kinase inhibitor, in human subjects who did not respond to the biologic, and/or in human subjects who responded to the biologic.

30. The method of claim 29, wherein the human subject will likely respond to therapy with the tyrosine kinase inhibitor when the activation level of full-length HER2 protein in the cellular extract is about 3-fold to about 7-fold higher than the median activation level of full-length HER2 protein.

31. The method of claim 28, wherein the activation level of full-length HER2 protein in the cellular extract is a ratio of the activation level of full-length HER2 protein in the cellular extract to an expression level of a control protein.

32. The method of claim 31, wherein the control protein is cytokeratin (CK).

33. The method of claim 32, wherein the human subject will likely respond to therapy with the tyrosine kinase inhibitor when the activation level of full-length HER2 protein in the cellular extract is higher than a reference activation level of full-length HER2 protein corresponding to a ratio of about 3.1 relative to the expression level of CK.

34. The method of claim 33, wherein the human subject will likely not respond to therapy with either the tyrosine kinase inhibitor or the biologic when the activation level of full-length HER2 protein in the cellular extract is lower than a reference activation level of full-length HER2 protein corresponding to a ratio of about 0.3 relative to the expression level of CK.

35-54. (canceled)

55. The method of claim 1, wherein the expression level of truncated HER2 protein, the expression level of full-length HER2 protein, and the activation level of full-length HER2 protein is determined.

56. The method of claim 1, wherein the expression level of full-length HER2 protein and the activation level of HER3 protein is determined.

57. The method of claim 1, wherein the method further comprises determining an expression level and/or an activation level of one or more additional signal transduction molecules in the cellular extract.

58. The method of claim 57, wherein the one or more additional signal transduction molecules is selected from the group consisting of AKT, PRAS40, ERK1 (MAPK3), ERK2 (MAPK1), RSK, and combinations thereof.

59. The method of claim 1, wherein the sample is a breast tumor tissue, whole blood, serum, or plasma sample.

60. The method of claim 59, wherein the breast tumor tissue sample is a needle biopsy sample.

61. The method of claim 1, wherein the expression level of truncated HER2 protein, the expression level of full-length HER2 protein, the activation level of full-length HER2 protein, the activation level of HER3 protein, and/or the activation level of PI3K protein is determined with Collaborative Enzyme Enhanced Reactive ImmunoAssay (CEER).

62. The method of claim 1, wherein the method further comprises administering the tyrosine kinase inhibitor or the biologic to the human subject.
